(19) United States
(12) Patent Application Publication
(10) Pub. No.: US 2023/0416184 A1
(43) Pub. Date: Dec. 28, 2023

(54) OPTIMIZED PROCESS FOR SYNTHESIZING ALKYL METHACRYLATE BY REDUCING UNWANTED BYPRODUCTS

(71) Applicant: Röhm GmbH, Darmstadt (DE)

(72) Inventors: Steffen KRILL, Muehltal (DE); Patrick Wings, Koeln (DE); Florian Klasovsky, Haltern am See (DE); Daniel Helmut Könlg, Stuttgart (DE)

(73) Assignee: Röhm GmbH, Darmstadt (DE)

(21) Appl. No.: 18/249,911

(22) PCT Filed: Oct. 6, 2021
(86) PCT No.: PCT/EP2021/077488

§ 371 Date: Apr. 20, 2023

(30) Foreign Application Priority Data
Oct. 23, 2020 (EP) 20203723.0

(51) Int. Cl. C07C 67/20; C07C 303/24; C07C 231/06; C07C 51/06; C07C 231/12

(52) U.S. Cl. CPC C07C 67/20; C07C 303/24; C07C 231/06; C07C 51/06; C07C 231/12

(57) ABSTRACT

An improved process for synthesizing alkyl methacrylates, in particular methyl methacrylate (MMA), involves reacting acetone cyanohydrin (ACH) and sulfuric acid in a first reaction stage (amidation). The process then involves heating the first reaction mixture in a second reaction stage (conversion) such that methacrylamide (MAA) is obtained; and then esterifying methacrylamide (MAA) with alcohol and water, preferably methanol and water, in a third reaction stage such that alkyl methacrylate is formed. The sulfuric acid used has a concentration of 98.0 wt % to 100.0 wt %. A subsequent working up of the third reaction mixture involves least two distillations in which the byproducts methacrylonitrile (MeAN) and acetone are obtained as an aqueous heteroazeotrope at least in part in the top fraction. At least some of the aqueous heteroazeotrope is removed from the process and at least partially reintroduced into the third reaction stage.

The present invention relates to an improved process for preparing alkyl methacrylates, especially methyl methacrylate (MMA), comprising the reaction of acetone cyanohydrin (ACH) and sulfuric acid in a first reaction stage (amidation) and the heating of the first reaction mixture in a second reaction stage (conversion) to obtain methacrylamide (MAA), and the subsequent esterification of methacrylamide (MAA) with alcohol and water, preferably methanol and water, in a third reaction stage (esterification) to form alkyl methacrylate, wherein the sulfuric acid used has a concentration in the range from 98.0% by weight to 100.0% by weight, and wherein the subsequent workup of the third reaction mixture comprises at least two distillation steps in which the methacrylonitrile (MAN) and acetone by-products are obtained at least partly in the tops fraction as a water-containing heteroazeotrope, wherein the water-containing heteroazeotrope is at least partly discharged from the process and at least partly recycled into the third reaction stage.

More particularly, the present invention relates to an optimized process for preparing alkyl methacrylate, comprising the specific adjustment and monitoring of the quality of the intermediates and products, especially MAA and MMA, wherein the formation of troublesome by-products, especially methacrylonitrile (MAN), acetone, methyl formate, methyl propionate and methyl isobutyrate (MIB), in the precursors and intermediates is reduced, and the yield of intermediates and products is improved.

### PRIOR ART

Methyl methacrylate (MMA) is used in large amounts for preparing polymers and copolymers with other polymerizable compounds. Furthermore, methyl methacrylate is an important monomer for various specialty esters based on the chemical synthon methacrylic acid (MA), which can be prepared by transesterification of MMA with the appropriate alcohol or are obtainable by condensation of methacrylic acid and an alcohol. There is consequently a great interest in very simple, economic and environmentally friendly processes for preparing this starting material.

The preparation of methacrylamide by the amidation (hydrolysis) of acetone cyanohydrin (ACH) is a widely employed process. For example, such an amidation, which is also referred to as ACH hydrolysis in the prior art, is an important intermediate step in the preparation of methyl methacrylate by what is called the ACH-sulfo process, wherein large amounts of sulfuric acid are used in the process. The preparation of methacrylic acid (MA) and methyl methacrylate (MMA) by the ACH-sulfo process is common knowledge and is described in the prior art, for example WO 2008/068064, WO 2013/143812, EP 0 226 724. In this process, in a first step, acetone cyanohydrin (ACH) is first prepared by reaction of hydrogen cyanide and acetone, which is then converted to methacrylamide (MAA). These steps are described in U.S. Pat. No. 7,253,307, EP 1 666 451 or EP 2007 059092 inter alia. The conversion of ACH to MAA (amidation) is brought about in a manner known to the person skilled in the art by a reaction between concentrated sulfuric acid and ACH. The reaction is exothermic, and so the heat of reaction is preferably removed rapidly from the system.

The conversion to MAA typically proceeds in two process steps. First of all, in the amidation step, an essentially anhydrous sulfuric acid solution comprising mainly alpha-hydroxyisobutyramide (HIBAm), the sulfate ester thereof alpha-sulfoxyisobutyramide (SIBA), and methacrylamide (MAA) (or protonated in salt form, in the form of the respective hydrogensulfates) is obtained. In the subsequent step, called the conversion, this solution is typically converted to methacrylamide (MAA) with β-elimination of water or sulfuric acid at high temperatures of 130° C. to 200° C. with usually short dwell times, for example about 15 min or less. Typically, after the conversion, the main MAA·H₂SO₄ product is present with a concentration in the solution of about 30% to 40% by weight (according to the sulfuric acid excess used).

The steps of amidation and of conversion, in terms of process technology, generally differ significantly in dwell time and also in the temperature level used. With regard to the chemical reaction, the amidation is typically conducted for a shorter period than the conversion and typically at lower temperatures than the conversion or subsequent esterification.

Document U.S. Pat. No. 4,529,816 describes a process for preparing alkyl methacrylates by the ACH-sulfo process, wherein the amidation is performed at temperatures around 100° C. with substantially superstoichiometric amounts of sulfuric acid (molar ratio of ACH:H₂SO₄ of about 1:1.3 to 1:1.8). It is pointed out that the sulfuric acid used should contain sufficient SO₃ to assure at least an acid strength of 98%, preferably at least 99.5%. More particularly, fuming sulfuric acid (oleum) should be used, which preferably has 101% sulfuric acid and hence free SO₃. In the subsequent step of the esterification, the reaction mixture is treated with an excess of water and alcohol at 100 to 150° C. U.S. Pat. No. 4,529,816 states that the methyl alpha-hydroxyisobutyrate (MHIB) by-product is separated from the methyl methacrylate product after the esterification and recycled into the process. In a similar manner, in the process according to U.S. Pat. No. 5,393,918, by-products including methyl alpha-hydroxyisobutyrate (MHIB) are isolated, dehydrated and recycled into the reaction. A disadvantage here is the need to isolate and recycle the MHIB by-product. It is additionally stated that the concentration of sulfuric acid in the conversion of ACH is not crucial.

Document DE 38 28 253 A1 describes a process for recycling spent sulfuric acid in the preparation of methacrylic esters by the ACH-sulfo process, wherein the spent acid, after the esterification, is concentrated, mixed with fresh acid and recycled. DE 38 28 253 A1 generally describes an acid strength of 96% to 101% in the reaction of acetone cyanohydrin with sulfuric acid.

Document DE 1 618 721 describes the reaction of acetone cyanohydrin (ACH) with sulfuric acid in two stages with a different ratio of sulfuric acid to ACH, by means of which the viscosity of the reaction mixture is to be controlled. In the process described in EP 0 226 724, the reaction is performed in the presence of an alkane solvent in order to control and to monitor the viscosity of the reaction mixture and the enthalpy of reaction.

Document CH 239749 describes a process for preparing methacrylamide by the action of sulfuric acid on acetone cyanohydrin at temperatures of 110 to 130° C. or 115 to 160° C., wherein 100% sulfuric acid, for example, is used.

U.S. Pat. No. 4,748,268 describes a process for esterifying methacrylic acid with a C₁-C₄ alcohol in the presence of a high-boiling organic liquid in a plug-flow reactor, in which the reaction mixture is continuously fractionated, wherein the distillate stream has a relatively high proportion of methacrylic ester and the bottom stream is recycled predominantly into the plug-flow reactor.

By-products formed in the amidation and conversion include carbon monoxide, acetone, sulfonation products of acetone, and cyclocondensation products of acetone with various intermediates. These by-products mentioned can usually be separated relatively effectively from the alkyl methacrylate product. In addition, however, depending on the reaction conditions, other by-products are formed, the separation of which from the alkyl methacrylate, especially from the methyl methacrylate product, is difficult or associated with considerable separation complexity. For example, the separation is found to be difficult on account of the azeotrope boiling points and the boiling points of the specific compounds. Troublesome by-products are especially methacrylonitrile (MAN), acetone, methyl isobutyrate (MIB) and methyl propionate (MP), and also diacetyl (di-AC, butane-2,3-dione). Methacrylonitrile (MAN) and acetone in particular are relevant troublesome by-products, the concentration of which in the recycle streams has to be monitored in order to assure uniform product quality in continuous operation.

Some of these troublesome by-products are responsible to a crucial degree for an elevated colour number in the alkyl methacrylate end product, especially MMA. The troublesome low molecular weight by-products may additionally make problems in the course of further polymerization and processing of the polymers, for example as a result of outgassing during extrusion or in injection moulding. Troublesome by-products having a double bond are polymerized into the polymer product as well as the alkyl methacrylate and impair the properties of the polymers, for example the transparency and haze characteristics when used in a moist air environment. In order to obtain on-spec alkyl methacrylate end product, especially MMA, the level of these by-products, such as MAN. MIB and/or MP, must be reduced in the reaction steps or they must be removed in the workup.

Methacrylonitrile (MAN) is typically formed as a by-product during the amidation reaction from acetone cyanohydrin (ACH) with elimination of water.

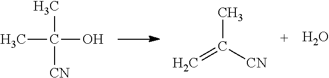

Methacrylonitrile (MAN) forms an azeotrope both with methanol (MeOH) and with methyl methacrylate (MMA), or has a similar boiling point as some azeotropes of methyl methacrylate, and can therefore be separated from the product only with difficulty and usually with considerable complexity. It is generally impossible to completely remove MAN with a reasonable level of cost and complexity.

On analysis of commercial MMA qualities based on ACH-sulfo technology, it is therefore notable that such a product quality always has greater or lesser traces of MAN, typically in an order of magnitude between 10 and 500 ppm (0.05% by weight in addition to MMA with a content of not less than 99.9% by weight). The simultaneous presence of water, methanol and acetone and various by-products also constitutes a particular challenge since, in the case of distillative removal, methanol and acetone as phase mediators often disrupt the formation of pure separation phases in particular concentration ranges and/or under particular physical conditions (pressure, temperature). Thus, formation of pure separation phases is often prevented, such that, after the phase separations, further separation and/or workup steps are frequently necessary. According to prior art, in the case of workup by the ACH-sulfo process, what are obtained are usually an organic MMA-containing phase and an aqueous methanol-containing phase, each of which contain the phase-mediating substances mentioned, especially acetone and methanol, and so further complex chemical engineering operations have to be used.

The amidation affords, as desired main products from the reaction, sulfoxyisobutyramide hydrogensulfate (SIBA·H₂SO₄) and methacrylamide hydrogensulfate (MAA·H₂SO₄) as a solution in excess sulfuric acid. Typically obtained additionally in the amidation solution is alpha-hydroxyisobutyramide hydrogensulfate (HIBAm·H₂SO₄), for example with a yield based on ACH of <5%. In the case of virtually full conversion of ACH, it is possible to obtain a yield of the above-described intermediates in the amidation step of typically about 94% to 96%. In this step, the above-described by-products are often formed in considerable amounts. The fundamental side reaction of this process step is the breakdown of ACH in the reaction matrix, which fundamentally also depends on the amidation temperature. Proceeding from HCN, this breakdown forms carbon monoxide which outgases out of the reaction solution. Acetone which is likewise formed is sulfonated and forms, for example, acetonedisulfonic acid (ADSA) and salts derived therefrom.

In addition, tar-like solid condensation products separate out of the ammonium hydrogensulfate- and sulfuric acid-containing process acid which is often regenerated in a sulfuric acid contact plant, and these hinder conveying of the process acid and have to be eliminated and disposed of with considerable cost and complexity. There is no description in the prior art of relationships between tar formation and reaction conditions, and there is thus a lack, for example, of pointers as to how the amount and consistency of tar formation can be positively influenced for the purposes of an undisrupted process regime.

It is also known that hydroxyisobutyric acid can be prepared proceeding from acetone cyanohydrin (ACH) by hydrolysis of the nitrile function in the presence of mineral acids. The prior art describes processes in which ACH is amidated and hydrolysed in the presence of water, wherein the hydroxyl function in the molecular complex is conserved at least in the first steps of the reaction; for example WO 2005/077878, JP H04 193845 A, JP S57 131736. For example, Japanese patent application JP S63-61932 states that ACH is hydrolysed in a two-stage process to hydroxyisobutyric acid, wherein ACH is first converted in the presence of 0.2 to 1.0 mol of water and 0.5 to 2 equivalents of sulfuric acid, forming the corresponding amide salts. These proposals for an alternative amidation in the presence of water, according to whether it is performed in the presence of methanol or without methanol, lead either to formation of methyl hydroxyisobutyrate (MHIB) or to formation of 2-hydroxyisobutyric acid (HIBAc). For example, according to JP S57 131736, the reaction of ACH with 0.8 to 1.25 equivalents of sulfuric acid is effected in the presence of less than 0.8 equivalent of water below 60° C., and then the reaction with more than 1.2 equivalents of methanol to give MHIB at temperatures of greater than 55° C.

None of the processes in the prior art describes a means of controlling and reducing the level of troublesome by-products, especially of methacrylonitrile (MAN) and acetone, which impair the properties of the alkyl methacrylate and of the alkyl methacrylate polymers to a particular degree and additionally reduce the overall yield of the process, since the effective removal of MMA therein is always associated with isolation losses of MMA.

More particularly, commercial MMA qualities having a purity of typically about 99.8% by weight contain, in a process-specific manner typical of ACH-sulfo technology, as well as MMA, methacrylonitrile (MAN) in the trace region, which can be determined analytically. In general, MAN is present in amounts of a few ppm to a few hundred ppm. The MAN content is often in the order of magnitude of 30 to 200 ppm and is subject to certain variations from batch to batch. In summary, it can be stated that the prior art does not give any pointers as to what relationships lead to formation of the unwanted by-products or how the levels of these can be reduced. Although it is known in principle to the person skilled in the art how MAN can be separated by distillation from pure MMA, this is possible only with impairment of the yield, or requires elevated apparatus complexity. There is thus a great need for an industrial means of influencing the formation of these troublesome by-products at the early stage of the reaction and reducing the level therein, such that the downstream separation steps are simplified. What would also be desirable would be methods that allow conversion of the MAN-containing streams of matter to products of value for the purpose of MMA production and/or effective discharge from the process.

### OBJECT OF THE INVENTION

It is an object of the invention to overcome the abovementioned disadvantages and to provide an improved process for preparing alkyl methacrylates based on the ACH-sulfo process, in which the amount of troublesome by-products, especially of methacrylonitrile (MAN) and acetone, can be reduced. This shall improve the product quality of the alkyl methacrylate and of the polymers and shaped bodies produced therefrom. In particular, there shall be an improvement in the processability and the mechanical and optical properties of the alkyl methacrylate polymers.

Moreover, by comparison with known processes, a comparable or elevated yield of alkyl methacrylate shall be obtained, for example with simultaneous reduction in losses of alkyl methacrylate in the workup and waste streams. It was a further object of the invention to provide a process that permits continuous treatment, in circulation operation, of MAN-containing workup streams in such a way that MAN can be hydrolysed to MAA and hence back to the desired MMA product.

Achievement of the Object

The object was achieved in that, in the process according to the invention, firstly the formation of the by-products mentioned is minimized by an optimized reaction regime in the alkyl methacrylate synthesis, and secondly troublesome by-products are removed as early as possible from the process via specific chemical sinks in the workup section and hence do not get into the end product. Chemical sinks are reactions in which a by-product, when recycled, is physically converted in such a way that constant buildup (a constant increase in concentration) in continuous operation is suppressed. In the best case, such a chemical sink that converts the by-product back to the desired target product is found.

It has been found that, surprisingly, the abovementioned objects are achieved by the process according to the invention. More particularly, it has been found that the amount of troublesome by-products, especially methacrylonitrile (MAN), acetone, methyl propionate and/or methyl isobutyrate, can be reduced when sulfuric acid containing no free SO₃, but especially containing small proportions of free water, is used in the conversion of acetone cyanohydrin (in amidation and conversion). It has been found to be particularly advantageous to use a sulfuric acid having a concentration in the range from 98.0% by weight to 100.0% by weight, preferably 99.0% by weight to 99.9% by weight.

It has additionally been found that the troublesome by-products, especially methacrylonitrile (MAN) and acetone, can be effectively discharged from the process to the degree required via an optimized workup of the reaction mixture after the esterification, comprising a suitable discharge and optimized circulation of process streams. More particularly, it has been found that an effective discharge of methacrylonitrile (MAN) and acetone can be achieved in that, in at least one azeotrope distillation step, the by-products are obtained at least partly in the tops fraction as a water-containing heteroazeotrope and are at least partly discharged from the process thereby, optionally after further separation steps. The heteroazeotrope comprising the troublesome by-products may optionally be separated into an aqueous phase and an organic phase, in which case the aqueous phase and/or the organic phase may be at least partly discharged from the process. It has surprisingly been possible here to remove the troublesome by-products from the process together with those streams of matter in which enrichment of the troublesome by-products is not to be expected on account of their physicochemical properties (especially water solubility and volatility). In addition, it is possible by combination of multiple distillation and extraction steps to discharge the troublesome by-products, especially acetone and methacrylonitrile, from the process as derivatives (e.g. acetone in sulfonated form), or to convert them to the target product (e g. MAN via MAA to MMA).

More particularly, it is possible with the aid of the process according to the invention to perform the industrial ACH-sulfo process more robustly, with lower propensity to faults and with higher yields, with the removal of alkyl methacrylates in the required quality being possible in an effective manner.

### DESCRIPTION OF THE INVENTION

The present invention relates to a process for preparing alkyl methacrylate, preferably methyl methacrylate, comprising
a. the reacting of acetone cyanohydrin and sulfuric acid in one or more reactors i in a first reaction stage (amidation) at a temperature in the range from 70 to 130° C. to obtain a first reaction mixture comprising sulfoxyisobutyramide and methacrylamide;
b. the converting of the first reaction mixture, comprising heating to a temperature in the range from 130 to 200° C., preferably 130 to 170° C., in one or more reactors II in a second reaction stage (conversion) to obtain a second reaction mixture comprising predominantly methacrylamide and sulfuric acid;
c. the reacting of the second reaction mixture with alcohol and water, preferably methanol and water, in one or more reactors III in a third reaction stage (esterification) to obtain a third reaction mixture comprising alkyl methacrylate, preferably methyl methacrylate;
d. and the separating of alkyl methacrylate from the third reaction mixture obtained from the third reaction stage;
wherein the sulfuric acid used in the first reaction stage has a concentration in the range from 98.0% by weight to 100.0% by weight, preferably 99.0% by weight to 99.9% by weight;
wherein the separation of alkyl methacrylate from the third reaction mixture comprises at least two distillation steps in which the methacrylonitrile and acetone by-products are obtained at least partly in the tops fraction as a water-containing heteroazeotrope,
wherein the water-containing heteroazeotrope comprising methacrylonitrile and acetone from at least one of these distillation steps is at least partly discharged from the process,
and wherein at least one stream comprising methacrylonitrile and acetone is at least partly recycled into the third reaction stage.

In the context of the present invention, the expression “ppm” without further qualifiers means ppm by weight (e.g. mg/kg).

The expression “stream, phase or fraction comprise a reactant, product and/or by-product” is understood in the context of the invention to mean that the compound(s) mentioned is/are present in the respective stream; for example, the predominant proportion of the reactant, product and/or by-product is to be found in the corresponding stream. In principle, further constituents may be present as well as the compounds mentioned. The naming of the constituents often serves to illustrate the respective process step.

The expression “vapour” or “vapour stream” in the context of the invention refers to a gaseous process stream, for example a gaseous top stream from a distillation column. Preferably, a gaseous vapour stream is liquefied after contact with a cooling device, for example a condenser, and can then form one or more liquid phases according to the composition, typically an aqueous phase and a predominantly organic phase.

First Reaction Stage (Amidation)

The process according to the invention comprises, as step a, the reacting of acetone cyanohydrin and sulfuric acid in one or more reactors I in a first reaction stage (amidation) at a temperature in the range from 70 to 130° C., preferably 70 to 120° C., more preferably 80 to 110° C., to obtain a first reaction mixture comprising sulfoxyisobutyramide and methacrylamide.

According to the invention, the sulfuric acid used in the first reaction stage has a concentration in the range from 98.0% by weight to 100.0% by weight, preferably of 99.0% by weight to 99.9% by weight, preferably of 99.3% to 99.9% by weight, especially preferably of 99.3% to 99.8% by weight. More particularly, the stated concentration of the sulfuric acid used is based on the total mass of the sulfuric acid feed stream (e.g. (2)). The use of a sulfuric acid having a zero content of free SO₃, especially a sulfuric acid with a water content of 0.1% to 0.5% by weight, has been found to be particularly advantageous. More particularly, it was thus possible to increase the amidation yield and reduce the proportion of by-products, especially MAN and acetone, especially at the early stage of the formation reaction or in various reaction stages.

The person skilled in the art is aware in principle of methods of determining the water content of streams of matter, for example of sulfuric acid feed streams. For example, the water content of streams of matter can be ascertained by mass balances, by measuring the density or speed of sound, by gas chromatography, by Karl Fischer titration, or by means of HPLC.

The acetone cyanohydrin (ACH) used can be prepared by means of known industrial processes (see, for example, Ullmanns Enzyklopädie der technischen Chemie [Ullmann's Encyclopedia of Industrial Chemistry], 4th edition, volume 7). Typically, hydrogen cyanide and acetone are converted to ACH in an exothermic reaction in the presence of a basic catalyst, for example an amine or alkali metal hydroxide. Such a process stage is described, for example, in DE 10 2006 058 250 and DE 10 2006 059 511.

Typically, the reaction (amidation) of acetone cyanohydrin (ACH) and sulfuric acid forms, as main products, alpha-hydroxyisobutyramide (HIBAm) or its hydrogensulfate (HIBAm·H₂SO₄), sulfuric esters of alpha-hydroxyisobutyramide (sulfoxyisobutyramide (SIBA)) or its hydrogensulfate (SIBA·H₂SO₄) and methacrylamide hydrogensulfate (MAA·H₂SO₄), as a solution in excess sulfuric acid.

In a preferred embodiment, the reaction mixture composed of acetone cyanohydrin (ACH) and sulfuric acid includes, in the first reaction mixture, a total amount of water in the range from 0.1 mol % to 20 mol %, especially 0.4 mol % to 10 mol %, based on the overall ACH supplied to the first reaction stage. Preference is given to using acetone cyanohydrin (ACH) in the first reaction stage, wherein the ACH or the ACH streams supplied (e.g. (1a) and/or (1b)) have an acetone content of not more than 9000 ppm, preferably of not more than 1000 ppm, based on the total amount of ACH which is supplied to the first reaction stage. Preferably, the ACH used has, or the ACH streams supplied (e.g. (1a) and/or (1b)) have, an ACH content of not less than 98% by weight, more preferably not less than 98.5% by weight, especially preferably not less than 99% by weight, based on the ACH streams supplied. Typically, the ACH stream supplied (e.g. (1a) and/or (1b)) contains 98.0% to 99.8% by weight, preferably 98.3% to 99.3% by weight, of acetone cyanohydrin, 0.1% to 1.5% by weight, preferably 0.2% to 1% by weight, of acetone, and 0.1% to 1.5% by weight, preferably 0.3% to 1% by weight, of water, based on the ACH stream.

Preferably, in the first reaction stage, acetone cyanohydrin (ACH) is used, wherein the ACH has, or the ACH streams supplied (e.g. (1a) and/or (1b)) have, a water content of 0.1 mol % to 10 mol %, especially 0.4 mol % to 5 mol %, based on the ACH present in the ACH streams supplied.

Preference is given to using an ACH quality which, as well as the pure substance, also contains the catalyst, but the catalyst is neutralized by a Brønsted acid, preferably sulfuric acid. Typically, the pH of the ACH used as feed stream is between pH 2 and pH 6. It is also possible for traces of HCN to be present in the ACH, but the content of HCN is monitored such that the concentration of HCN in the ACH does not exceed 2000 ppm, preferably does not exceed 1000 ppm; more preferably, the HCN content is monitored such that it is between 100 ppm and 800 ppm. This can be effected by stripping and distillation, with ACH as bottom stream being freed of HCN very substantially within the limits described.

The first reaction stage is preferably conducted with an excess of sulfuric acid. The sulfuric acid preferably serves as solvent. At the same time, the sulfuric acid serves as reactant for the preparation of the SIBA intermediate and as catalyst for the amidation. The sulfuric acid excess can especially serve to keep the viscosity of the reaction mixture low, which can assure faster removal of heat of reaction and a lower temperature of the reaction mixture. This can especially bring distinct yield benefits. Even though viscosity and dissolution capacity are improved with more sulfuric acid, which ultimately entails an elevated yield and selectivity, the upper limit in the amount of sulfuric acid used is limited for economic reasons since the resulting volume of waste acid has to be recycled or processed further.

Preference is given to using sulfuric acid and acetone cyanohydrin (ACH) in the first reaction stage, the amidation, in a molar ratio of sulfuric acid to ACH in the range from 1.2 to 2; preferably 1.25 to 1.6; more preferably of 1.4 to 1.45. Preference is given to using two or more reactors I in the first reaction stage, in which case sulfuric acid and acetone cyanohydrin (ACH) are used in the first reactor I in a molar ratio of sulfuric acid to ACH in the range from 1.6 to 3; preferably 1.7 to 2.6; more preferably 1.8 to 2.3; and wherein sulfuric acid and acetone cyanohydrin (ACH) are used in the last reactor I (for example in the second reactor I) in a molar ratio of sulfuric acid to ACH in the range from 1.2 to 2.0; preferably from 1.2 to 1.8; especially preferably from 1.3 to 1.7. An optimal compromise between achievable yield and sulfuric acid consumption is found to be a preferred molar ratio of 1.25 to 1.6, which can ultimately be assessed from the standpoint of economic optimization. Typically, more sulfuric acid increases the costs for this feedstock and the expenditure required for the disposal of the resulting waste acid mixture, but the yield based on ACH can generally be increased slightly once again.

The reaction of acetone cyanohydrin with sulfuric acid in the first reaction stage is exothermic. It is therefore advantageous to largely or at least partly remove the heat of reaction obtained, for example with the aid of suitable heat exchangers, in order to obtain an improved yield. Since the viscosity of the reaction mixture rises significantly with falling temperature, and hence circulation, flow and heat exchange in the reactors I are limited, excessive cooling should be avoided, however. Furthermore, there can be partial or complete crystallization of ingredients on the heat exchangers at low temperatures in the first reaction mixture, which can lead to abrasion, for example in the pump housings, pipelines and heat exchanger tubes of the reactors i. This so-called sulfation should preferably be avoided at all costs since it requires shutdown of the plant and cleaning of the reactor.

For cooling of the reactor circuits, it is possible in principle to use known and suitable cooling media. It is advantageous to use cooling water. Typically, the cooling medium, especially the water, has a temperature below the process conditions chosen. Advantageously, the cooling medium, especially the cooling water, has a temperature in the range from 20 to 90° C., preferably from 50 to 90° C. and more preferably from 70 to 80° C.

To stop the temperature from going below the crystallization point of methacrylamide, the heat exchanger (reactor cooler) is typically operated with a hot water secondary circuit. Preference is given here to temperature differences in the inlet/outlet of the apparatus on the product side of about 1 to 20° C., especially 2 to 10° C.

The conversion of acetone cyanohydrin and sulfuric acid in one or more reactors I in a first reaction stage (amidation) is effected at a temperature in the range from 70 to 130° C., preferably from 80 to 120° C., more preferably from 90 to 110° C. The amidation in the first reaction stage in the reactor I or in multiple reactors I is often conducted at standard pressure or slightly elevated pressure.

Typically, the first reaction stage (amidation) can be performed batchwise and/or continuously. Typically, the first reaction stage can be executed in a stirred tank, a stirred tank cascade or a loop reactor, or a combination of these apparatuses. The first reaction stage is preferably conducted continuously, for example in one or more loop reactors. Suitable reactors and processes are described, for example, in WO 2013/143812. Advantageously, the first reaction stage can be conducted in a cascade of two or more loop reactors. Especially preferably, the reaction in the first reaction stage is effected in one or more (preferably two) loop reactors.

The first loop reactor is typically operated at a circulation ratio (ratio of circulation volume flow rate to feed volume flow rate) in the range from 5 to 110, preferably 10 to 90, more preferably 10 to 70. In a subsequent loop reactor, the circulation ratio is preferably within a range from 5 to 100, preferably from 10 to 90, more preferably from 10 to 70.

Typically, the static dwell time in the reactors I, especially in the loop reactors I, is in the range from 5 to 35 minutes, preferably from 8 to 20 minutes.

A suitable loop reactor preferably has the following elements: one or more addition points for ACH, one or more addition points for sulfuric acid, one or more gas separators, one or more heat exchangers, one or more mixers, and a pump. The mixers are frequently executed as static mixers.

The ACH can be added in principle at any point to the one or more reactors I (e.g. loop reactors). However, it has been found to be advantageous when the ACH is added at a well-mixed site. Preference is given to adding the ACH to a mixing element, for example to a mixer having moving parts, or to a static mixer.

The sulfuric acid can be added in principle at any point to the one or more reactors I (e.g. loop reactors). The sulfuric acid is preferably added upstream of the addition of the ACH. Particular preference is given to adding the sulfuric acid on the suction side of the respective reactor pump. It is often possible thereby to improve the pumpability of the gas-containing reaction mixture.

The reactors I (e.g. loop reactors i) preferably each include at least one gas separator. Typically, it is possible to withdraw product stream (first reaction mixture) continuously via the gas separator on the one hand; on the other hand, it is possible to remove and discharge gaseous by-products. Typically, the gaseous by-product formed is mainly carbon monoxide. Preference is given to guiding a portion of the offgas which is obtained in the amidation into a gas separator together with the second reaction mixture which is obtained in the second reaction stage (conversion).

In a preferred embodiment, the first reaction stage comprises the reaction of acetone cyanohydrin (ACH) and sulfuric acid in at least two separate reaction zones, preferably in at least two loop reactors.

Preference is given to reacting acetone cyanohydrin (ACH) and sulfuric acid in such a way that the reaction volume is divided into at least two reaction zones, and the total amount of ACH is metered separately into the different reaction zones. The amount of ACH which is supplied to the first reactor or to the first reaction zone is preferably not less than the amounts of ACH that are supplied to the downstream reactors or to the downstream reaction zones.

Preference is given to introducing 50% to 90% by weight, preferably 60% to 75% by weight, of the total volume flow rate of ACH supplied into the first reactor (e.g. (1a)). The remaining amount of ACH supplied is introduced into the second reactor and optionally into further reactors (e.g. (1b)). Typically, the total amount of ACH is divided between the first reactor I (e.g. (A)) and the second reactor I (e.g. (B)) in a mass ratio of first reactor I:second reactor I in the range from 70:30 to 80:20, preferably of about 75:25.

The molar ratio of added sulfuric acid to ACH in the first reactor or in the first reaction zone is greater than the corresponding molar ratio in the downstream reactors or in the downstream reaction zones.

Especially preferably, the first reaction stage comprises the reaction of acetone cyanohydrin (ACH) and sulfuric acid in at least two separate reactors, preferably at least two loop reactors, wherein sulfuric acid and acetone cyanohydrin (ACH) are used in the first reactor in a molar ratio of sulfuric acid to ACH in the range from 1.6 to 3; preferably 1.7 to 2.6; especially preferably 1.8 to 2.3, and wherein sulfuric acid and acetone cyanohydrin (ACH) are used in the second reactor in a molar ratio of sulfuric acid to ACH in the range from 1.2 to 2.0; preferably from 1.2 to 1.7; especially preferably from 1.3 to 1.7.

In a particularly preferred embodiment, the conversion in the first reaction stage is effected in two or more loop reactors (e.g. (A) and (B)), in which case the total amount of ACH is metered into the first and at least one further loop reactor. Especially preferably, each loop reactor comprises at least one pump, a heat exchanger cooled with water as medium, a gas separation apparatus, at least one gas conduit connected to the gas separation apparatus, and at least one feed conduit for ACH in liquid form. Preferably, the at least two loop reactors are connected to one another in such a way that the entire resulting reaction mixture from the first reactor is guided into the downstream reactors, and the reaction mixture in the downstream reactors is admixed with further liquid ACH and optionally further amounts of sulfuric acid.

Typically, after the first reaction stage (amidation), a first reaction mixture is obtained, containing 5% to 25% by weight of sulfoxyisobutyramide (SIBA), 5% to 25% by weight of methacrylamide (MAA) and <3% hydroxyisobutyramide (HIBAm), based in each case on the overall reaction mixture, dissolved in the sulfuric acid reaction matrix.

Second Reaction Stage (Conversion)

The process according to the invention comprises, in step b, the converting of the first reaction mixture, comprising heating to a temperature in the range from 130 to 200° C., preferably 130 to 180° C., preferably 130 to 170° C., particularly preferably 140 to 170° C., in one or more reactors II in a second reaction stage (conversion) to obtain a second reaction mixture comprising predominantly methacrylamide (MAA) and sulfuric acid.

Typically, when heating the first reaction mixture (conversion), which is a sulfuric acid solution comprising SIBA, HIBAm and MAA, each predominantly in the form of the hydrogensulfates, to a temperature in the range from 130 to 200° C., preferably 130 to 180° C., the amount of MAA or MAA·H₂SO₄ is increased by dehydration of the HIBAm or SIBA.

Especially preferably, the conversion in the second reaction stage is effected at a temperature in the range from 130 to 200° C., preferably from 130 to 180° C., more preferably 140 to 170° C., and a dwell time in the range from 2 to 30 minutes, preferably 3 to 20 minutes, especially preferably 5 to 20 minutes.

The conversion reaction can typically be divided into two sections, wherein the amidation mixture is raised relatively quickly to the required conversion temperature in the first part, and wherein the mixture, having attained the reaction temperature, is kept virtually adiabatic until the desired conversion in the second part. Typically, the reaction is optimized so as to maximize the yield of MAA, and such that SIBA has been depleted apart from traces, and HIBAm is likewise present only in the trace region of a few hundred or a few thousand ppm.

Preference is given to heating in the second reaction stage (conversion) over a minimum period of time. In particular, the heating in the second reaction stage is effected for a period of 1 to 30 minutes, preferably 1 to 20 minutes, more preferably 2 to 15 minutes, most preferably 2 to 10 minutes. In a preferred embodiment, the second reaction stage (conversion) comprises the heating of the reaction mixture, for example in one or more preheater segments, and the guiding of the reaction mixture under approximately adiabatic conditions, for example in one or more dwell segments.

The conversion can be conducted in known reactors that enable the attainment of the temperatures mentioned within the periods of time mentioned. The energy can be supplied here in a known manner, for example by means of steam, hot water, suitable heat transfer media, electrical energy or electromagnetic radiation, such as microwave radiation. Preference is given to conducting the conversion in the second reaction stage in one or more heat exchangers.

In a preferred embodiment, the conversion in the second reaction stage is conducted in a heat exchanger comprising a two-stage or multistage arrangement of pipe coils. The multistage pipe coils are preferably arranged in opposing rotations.

The heat exchanger may be combined, for example, with one or more gas separators. For example, it is possible to guide the reaction mixture through a gas separator after it has left the first pipe coil of the heat exchanger and/or after it has left the second pipe coil of the heat exchanger. It is especially possible here to separate gaseous by-products from the reaction mixture.

Typically, the first reaction mixture obtained in the first reaction stage is guided completely into the reactor II of the second reaction stage. Optionally, the steps of amidation and conversion can be performed alternately, in which case the conversion is preferably the last step before the subsequent third reaction stage (e.g. esterification). Preferably, this embodiment comprises an intermediate conversion between two amidation steps and a final conversion. Preferably, the second reaction mixture obtained in the second reaction stage is guided into a second reactor I for a further amidation step.

Preferably, the second reaction mixture which is obtained after the conversion is guided into a gas separator (e.g. (D)), wherein gaseous by-products can be at least partly separated from the second reaction mixture. Typically, the degassed second reaction mixture is guided fully into the third reaction stage (esterification). Preferably, the offgas which is obtained after the conversion in the gas separator is discharged fully or partly from the process. Alternatively, the offgas which is obtained after the conversion in the gas separator is guided fully or partly into the third reaction stage (esterification). In this variant, gaseous organic components present alongside the main by-product of the amidation and conversion, namely CO, are introduced into the esterification reaction; in this way, the gas can serve as stripping medium for crude MMA. MAN is one such component which can get into the esterification reaction in gaseous form in this way, where it is partly depleted to MAA.

More particularly, the process according to the invention enables reduction in the amount of troublesome by-products, preferably in the amounts of MAN, acetone, MA and/or HIBAm, in the second reaction mixture (after amidation and conversion). Preferably, the second reaction mixture contains not more than 3% by weight, preferably not more than 2% by weight, of MA, not more than 1.5% by weight, preferably not more than 1% by weight, of HIBAm, and not more than 0.3% by weight of MAN, based in each case on the overall second reaction mixture.

Preferably, the second reaction mixture (after amidation and conversion) contains 30% to 40% by weight of methacrylamide (MAA), based on the overall second reaction mixture. Preferably, the second reaction mixture (after amidation and conversion) contains 30% to 40% by weight of MAA, 0% to 3% by weight of MA and 0.2% to 1.5% by weight, preferably 0.2% to 1% by weight, of HIBAm and 0.01% to 0.3% by weight of MAN, based in each case on the overall second reaction mixture. The second reaction mixture (i.e. after the conversion) thus contains a significantly lower concentration of HIBAm than the first reaction mixture (i.e. directly after the amidation).

Third Reaction Stage (Esterification)

The process according to the invention comprises, in step c, the reacting of the second reaction mixture comprising predominantly methacrylamide with alcohol and water, preferably with methanol and water, in one or more reactors III in a third reaction stage (esterification) to obtain a third reaction mixture comprising alkyl methacrylate.

The conditions for the esterification on an industrial scale are known to the person skilled in the art and are described, for example, in U.S. Pat. No. 5,393,918.

The conversion in the third reaction stage (esterification) is preferably conducted in one or more suitable reactors, for example in heated tanks. In particular, it is possible to use steam-heated tanks. In a preferred embodiment, the esterification is effected in two or more, for example three or four, successive tanks (tank cascade).

Typically, the esterification is conducted at temperatures in the range from 90 to 180° C., preferably from 100 to 150° C., at pressures up to 7 bara, preferably of not more than 2 bara, and using sulfuric acid as catalyst. It is particularly preferable to use the sulfuric acid from the second reaction mixture as catalyst and not to use additional acid over and above this amount.

Preference is given to reacting the second reaction mixture with at least equimolar amounts or an excess of alcohol and water, preferably an excess of methanol and water. The addition of the second reaction mixture comprising predominantly methacrylamide and the addition of alcohol are preferably effected in such a way as to result in a molar ratio of methacrylamide to alcohol in the range from 1:0.7 to 1:1.6. In a preferred embodiment, the reaction in the third reaction stage is effected in two or more reactors III, in which case there is a molar ratio of methacrylamide to alcohol in the first reactor III in the range from 1:0.7 to 1:1.4, preferably in the range from 1:0.9 to 1:1.3, and in which case there is a molar ratio of methacrylamide to alcohol in the second and possible downstream reactors III in the range from 1:1.0 to 1:1.3.

Preferably, the alcohol supplied to the third reaction stage (esterification) is composed of alcohol freshly supplied to the process (fresh alcohol) and of alcohol present in recycled streams (recycling streams) in the process according to the invention. It is additionally possible in the process according to the invention to use alcohol present in recycling streams from downstream processes.

The alcohol may especially be selected from linear, branched, saturated and unsaturated C₁-C₈ alcohols, preferably C₁-C₄ alcohols. More particularly, the alcohol is a saturated C₁-C₄ alcohol. The alcohol is preferably selected from methanol, ethanol, propanol and butanol. The alcohol is more preferably methanol.

Typically, water is added to the reactor III or to the reactors III of third reaction stage in such a way that the concentration of water is in the range from 10% to 30% by weight, preferably 15% to 25% by weight, based in each case on the overall reaction mixture in the reactor III.

In principle, the water supplied to the third reaction stage (esterification) may come from any source and may contain various organic compounds, provided that no compounds are present that have an adverse effect on the esterification or the downstream process stages. The water supplied to the third reaction stage preferably comes from recycled streams (recycling streams) in the process according to the invention, for example from the purification of the alkyl methacrylate. It is additionally possible to supply fresh water, especially demineralized water or well water, to the third reaction stage (esterification).

Esterification with methanol typically affords a third reaction mixture comprising alkyl methacrylate (especially MMA), methyl hydroxyisobutyrate (MHIB) and further above-described by-products, and also significant amounts of water and unconverted alcohol (especially methanol).

In a preferred embodiment, the esterification is effected in two or more (especially three or four) successive tanks (tank cascade), wherein the liquid overflow and the gaseous products are guided from the first tank into the second tank. The corresponding procedure is typically followed with possible downstream tanks. More particularly, such a mode of operation can reduce foam formation in the tanks. In the second tank and in the possible downstream tanks, it is likewise possible to add alcohol. The amount of alcohol added here is preferably at least 10% less compared to the preceding tank. The added alcohol may typically be fresh alcohol and/or recycled alcohol-containing streams. The concentration of water in the various tanks may typically be different. The temperature of the second reaction mixture fed into the first tank is typically in the range from 100 to 180° C. The temperature in the first tank is typically in the range from 90 to 180° C., and the temperature in the second and in the possible downstream tanks is in the range from 100 to 150° C.

In a preferred embodiment, the evaporable fraction of the third reaction mixture which is obtained in the third reaction stage is removed from the reactors III in gaseous form (vapour) and sent to further workup, for example a distillation step. More particularly, the evaporable fraction of the third reaction mixture can be guided in the form of vapour into the bottom of a downstream distillation column K1 (primary column K1). If a cascade consisting of multiple reactors III, for example multiple stirred tanks, is used, it is possible to remove the evaporable fraction of the resultant reaction mixture as a vapour stream in each tank and guide it to further workup. Optionally, only the evaporable fraction of the reaction mixture formed in the last tank (as the third reaction mixture) is removed as vapour stream and guided to further workup. This vapour stream formed in the esterification (third reaction mixture) is typically an azeotropic mixture comprising water, alkyl methacrylate, alcohol and the by-products described, e.g. methacrylonitrile and acetone. Typically, this vapour stream formed in the esterification (third reaction mixture) has a temperature in the range from 60 to 120° C., where the temperature depends on the alcohol used. Typically, this vapour stream formed in the esterification has a temperature in the range from 70 to 90° C. if methanol is used as alcohol.

It is advantageously possible to add one or more stabilizers in various streams of the process according to the invention in order to prevent or reduce polymerization of the alkyl methacrylate. For example, it is possible to add a stabilizer to the third reaction mixture obtained after the esterification. It is further advantageous to add a stabilizer to the tops fraction from the first distillation step K1 (primary column K1). It is preferably possible to use phenothiazine and other equivalent stabilizers, for example in the first reaction stage (amidation) and/or in the second reaction stage (conversion). It is also possible with preference to use phenolic compounds, quinones and catechols in the third reaction stage (esterification) and/or in the workup section. Additionally used are amine N-oxides, for example TEMPOL, or combinations of the stabilizers mentioned. Particular preference is given to mixtures of at least two of these stabilizers that are added at various points in the process.

A waste stream (e.g. (11)) consisting essentially of dilute sulfuric acid and ammonium hydrogensulfate is preferably removed from the third reaction stage (esterification). This waste stream is typically discharged from the process. This waste stream, especially together with one or more aqueous waste streams from the process according to the invention, is preferably sent to a process for regeneration of sulfuric acid or a process for obtaining ammonium sulfate.

Workup of the Third Reaction Mixture

The process according to the invention comprises, in step d, the separating of alkyl methacrylate from the third reaction mixture, wherein the separation (workup) of alkyl methacrylate from the third reaction mixture comprises at least two distillation steps in which the methacrylonitrile (MAN) and acetone by-products are obtained at least partly as a water-containing heteroazeotrope in the tops fraction and are especially at least partly separated from the alkyl methacrylate, wherein the water-containing heteroazeotrope comprising methacrylonitrile (MAN) and acetone is discharged at least partly from the process from at least one of these distillation steps, and wherein at least one stream comprising methacrylonitrile and acetone is at least partly recycled into the third reaction stage.

The at least one stream comprising methacrylonitrile and acetone which is at least partly recycled into the third reaction stage (esterification) is preferably water-containing heteroazeotrope comprising methacrylonitrile and acetone from at least one of the distillation steps, as described above.

For example, the aqueous phase and/or the organic phase of the water-containing heteroazeotrope may be discharged from the process from at least one distillation step and/or mixtures thereof, optionally after further workup steps, such as condensation, phase separation, extraction and scrubbing steps.

Preferably, at least one aqueous phase which is obtained by means of condensation and phase separation of the water-containing heteroazeotrope from at least one of the distillation steps is recycled fully or partly, optionally after an extraction step, into the third reaction stage (esterification), where it is contacted with the second reaction mixture comprising predominantly methacrylamide and sulfuric acid.

Preferably, at least one aqueous phase which is obtained by means of condensation and phase separation of the water-containing heteroazeotrope from at least one of the distillation steps is discharged fully or partly from the process, optionally after an extraction step.

The separation of alkyl methacrylate from the third reaction mixture (step d) preferably comprises at least one phase separation step in which the water-containing heteroazeotrope from at least one of the distillation steps is separated into an aqueous phase comprising methacrylonitrile and acetone and an organic phase comprising predominantly alkyl methacrylate, wherein the aqueous phase is discharged fully or partly from the process.

In a further preferred embodiment, the water-containing heteroazeotrope from at least one of the distillation steps is discharged fully or partly from the process, at least partly in the form of a gaseous stream, optionally after a scrubbing step. For example, the water-containing heteroazeotrope from at least one distillation step can be removed in the form of a vapour stream and discharged from the process in gaseous form (as an offgas stream), optionally after further workup steps, for example selected from condensation, phase separation, extraction and scrubbing steps.

Preferably, the separation of alkyl methacrylate from the third reaction mixture (step d) comprises at least one phase separation step in which the water-containing heteroazeotrope from at least one of the distillation steps is separated into an aqueous phase comprising methacrylonitrile and acetone, and an organic phase comprising predominantly alkyl methacrylate, wherein the aqueous phase is partly discharged from the process and/or partly recycled into the third reaction stage, and wherein the organic phase comprising predominantly alkyl methacrylate is recycled fully or partly into the at least one distillation step.

As well as the troublesome MAN and acetone by-products, the water-containing heteroazeotrope which is obtained as tops fraction in the at least one distillation step typically comprises alcohol, for example methanol, water, dimethyl ether and methyl formate.

Methacrylonitrile (MAN) forms an azeotrope both with methanol (MeOH) and with methyl methacrylate (MMA), or has a similar boiling point with some azeotropes of methyl methacrylate, and can therefore be separated from the product only with difficulty and usually with considerable complexity. Typically, the troublesome MAN by-product, in the at least one distillation step as described above, is therefore typically obtained both in the tops fraction as water-containing heteroazeotrope and in the bottoms fraction.

Primary Column (K1) and Prepurification

The removal of alkyl methacrylate in step d of the process according to the invention preferably comprises the prepurification of the third reaction mixture which is obtained in the esterification. More particularly, the prepurification comprises at least one distillation step K1 (e.g. primary column (F)), at least one phase separation step (e.g. phase separator I, (G)) and at least one extraction step (e.g. extraction step (H)). In a further embodiment, the prepurification comprises at least two distillation steps, e.g. primary column K1 and primary stripper column K4 (e.g. (1)), and at least one phase separation step (e.g. phase separator (K)).

Preferably, the third reaction mixture obtained in the third reaction stage is evaporated continuously, wherein the resultant vapour stream (e.g. (12)) is fed to a first distillation step K1 (e.g. primary column (F)) in which a tops fraction (e.g. (14a) or (14b)) comprising alkyl methacrylate, water and alcohol, and a bottoms fraction (e.g. (13)) comprising higher-boiling components are obtained, and wherein the bottoms fraction is recycled fully or partly into the third reaction stage. More particularly, the tops fraction of the distillation step K-1 (e.g. (14a) or (14b)) is a water-containing heteroazeotrope comprising methacrylonitrile and acetone.

In a preferred embodiment (variant A), the tops fraction of the distillation step K1 (e.g. (14a)) comprising alkyl methacrylate, water and alcohol is separated in a phase separation step (phase separator i, e.g. (G)) into an organic phase OP-1 (e.g. (15a)) comprising the predominant portion of the alkyl methacrylate and an aqueous phase WP-1 (e.g. (15b)) comprising alcohol and further water-soluble compounds, with the aqueous phase typically being recycled fully or partly into the third reaction stage. Further preferably, the organic phase OP-1 comprising the predominant portion of the alkyl methacrylate is subjected to an extraction (e.g. (H)), preferably using water as extractant, wherein the aqueous phase from this extraction (e.g. 17b) is typically recycled fully or partly into the third reaction stage (esterification).

In a further preferred embodiment (variant B), the tops fraction from distillation step K1 (e.g. (14b)) comprising alkyl methacrylate, water and alcohol is guided as vapour stream into a further distillation step K4 (e.g. primary stripper column (I)), in which a water-containing heteroazeotrope (e.g. (19a)) comprising methacrylonitrile and acetone is obtained as tops fraction, and a bottoms fraction comprising alkyl methacrylate. Preferably, the tops fraction (e.g. (19a)) from distillation step K4, optionally after a scrubbing step (e.g. (J)), preferably after a scrubbing step with alcohol (e.g. methanol), is discharged fully or partly from the process in the form of a gaseous stream (e.g. (21a)). The bottoms fraction from distillation step K4 is preferably separated in a phase separation step (phase separator II, e.g. (K)) into an aqueous phase WP-2 (e.g. (20b)) comprising methacrylonitrile and acetone, and an organic phase OP-2 (e.g. (20a)) comprising the predominant portion of the alkyl methacrylate. Typically, the aqueous phase WP-2 comprising methacrylonitrile and acetone is recycled fully or partly into the third reaction stage (esterification).

Azeotrope Column (K2) and Purifying Column (K3)

The separation of alkyl methacrylate from the third reaction mixture (step d) preferably comprises guiding an organic phase (e.g. (17a) from extraction (H) or (20a) from phase separator (K)) comprising the predominant portion of the alkyl methacrylate into a distillation step K2 (azeotrope column, e.g. (L)) in which the tops fraction (e.g. (22a)) obtained is a water-containing heteroazeotrope comprising methacrylonitrile and acetone, and the bottoms fraction obtained is a crude alkyl methacrylate product (e.g. (22b)).

Preference is given to conducting distillation step K2 (azeotrope column, e.g. (L)) under reduced pressure. Preference is given to preheating the organic feed of distillation step K2 (e.g. (17a) or (20a)) and guiding it to the top of distillation column K2. It is typically possible to heat the top of the column indirectly with low-pressure steam by means of an evaporator.

Preference is given to removing a water-containing heteroazeotrope (e.g. (22a)) comprising alkyl methacrylate (e.g. MMA), water, alcohol (especially methanol), acetone, methacrylonitrile and further low boilers at the top of distillation column K2 (azeotrope column, e.g. (L)).

Typically, a bottoms fraction (e.g. (22b)) comprising the predominant proportion of the alkyl methacrylate, especially methyl methacrylate, and which is virtually free of low boilers, but contaminated with high boilers, for example methacrylic acid (MA) and methyl hydroxyisobutyrate (MHIB), is obtained in distillation step K2 (azeotrope column, e.g. (L)).

The crude alkyl methacrylate product (e.g. (22b)) which is obtained as bottoms fraction from distillation step K2 (azeotrope distillation, e.g. (L)) preferably contains at least 99.0% by weight of alkyl methacrylate. The crude alkyl methacrylate product (e.g. (22b)) which is obtained as bottoms fraction from distillation step K2 (azeotrope distillation, e.g. (L)) preferably has a MAN content of 20 to 2000 ppm.

In a preferred embodiment, the tops fraction (e.g. (22a)) from distillation step K2 (azeotrope column, e.g. (L)) is first guided as vapour stream into a condenser (e.g. (M)) and condensed stepwise under reduced pressure. This stepwise condensation preferably gives rise to a biphasic condensate I (e.g. (23a)) in the first stage (on the suction side of the condenser), and a further condensate II (e.g. 23 (d)) in the second stage (on the pressure side of the condenser). The offgas (e.g. (23e) or (23b)) formed in the stepwise condensation (especially in the condensation on the pressure side) is preferably discharged from the process, optionally after a scrubbing step (e.g. (J)).

In a preferred embodiment (variant A), the biphasic condensate I (e.g. (23a)) from the first stage of the condensation is guided into a phase separator (e.g. (N)), and the further condensate II (e.g. 23 (d)) from the second stage of the condensation is used as extractant in a downstream extraction step.

In another preferred embodiment (variant B), the liquid phases from the stepwise condensation (e.g. (M)) are combined and guided into a phase separator (e.g. (K)) in the form of a liquid biphasic stream (e.g. (23c)).

The water-containing heteroazeotrope which is obtained as tops fraction in distillation step K2 (e.g. (L)), typically after condensation (e.g. in (M)), is preferably separated in a phase separator II (e.g. in phase separator (N) or (K)) into at least one organic phase OP-2 comprising alkyl methacrylate and at least one aqueous phase WP-2 comprising MAN, acetone and methanol. The aqueous phase WP-2 and/or the organic phase OP-2 is preferably discharged fully or partly from the process. In particular, the aqueous phase WP-2 (e.g. (20b) or (24b)) is recycled fully or partly into the third reaction stage (esterification) (e.g. (26c) or (20b)), typically after a phase separation (e.g. in (N) or (K)). Especially preferably, the aqueous phase WP-2 comprising methacrylonitrile (MAN) and acetone is partly discharged from the process and partly recycled into the third reaction stage (esterification).

The aqueous phase WP-2 (e.g. (20b) or (24b)) often contains 10 to 10 000 ppm of MAN, based on the overall aqueous phase WP-2.

Preferably, the organic phase OP-2 of the water-containing heteroazeotrope which is obtained as tops fraction in distillation step K2 is recycled fully or partly, preferably fully, into distillation step K2 (e.g. (24a) or (20a)), typically after a phase separation (for example in (N) or (K)). In particular, the organic phase OP-2 (e.g. (24a) or (20a)) comprises alkyl methacrylate and methacrylonitrile (MAN).

Typically, the predominant proportion of MAN present in the tops fraction from distillation step K2 is to be found in the organic phase (OP-2) of the heteroazeotrope. Preferably, the complete or partial recycling of the organic phase of the heteroazeotrope (OP-2) into distillation step K2 (e.g. (L)) can achieve enrichment of the troublesome by-products, especially MAN, and hence more effective removal, for example via the aqueous phase of the heteroazeotrope (WP-2).

In a preferred embodiment, the weight ratio of the total amount of MAN which is recycled into the process, preferably into the third reaction stage (esterification) (e.g. via (28a)), to the total amount of MAN which is discharged from the process (e.g. via (28b)) is less than 7, preferably less than 5, especially less than 3. This index indicates the importance of the recycling or circulation for chemical conversion or removal of MAN. Typically, there will otherwise be an increase in the MAN concentration in the end product (e.g. MMA), with the consequence that a further processing step, e.g. a further distillation, would have to be conducted, which is undesirable.

In a preferred embodiment, the crude alkyl methacrylate product (e.g. (22b)) from distillation step K2 is guided into a further distillation step K3 (purifying column) in which the alkyl methacrylate is separated from higher-boiling compounds, and in which the tops fraction obtained (e.g. (25a)) is a pure alkyl methacrylate product. Preferably, the pure alkyl methacrylate product (e.g. (25a)) from distillation step K3 contains at least 99.9% by weight, preferably at least 99.95% by weight, based on the pure alkyl methacrylate product, of alkyl methacrylate. Preferably, the pure alkyl methacrylate product (e.g. (25a)) from distillation step K3 contains a content of methacrylonitrile (MAN) in the range from 10 to 300 ppm, preferably 10 to 100 ppm, more preferably 10 to 80 ppm, especially preferably 50 to 80 ppm, based on the pure alkyl methacrylate product. The pure alkyl methacrylate product preferably has a content of acetone of not more than 10 ppm, preferably of not more than 2 ppm, more preferably of not more than 1 ppm, based on the pure alkyl methacrylate product.

In a preferred embodiment, in the second distillation step K2 (azeotrope column) (e.g. (L)), the bottoms fraction obtained is a crude alkyl methacrylate product (e.g. (22b)) preferably containing at least 99.0% by weight of alkyl methacrylate, wherein the crude alkyl methacrylate product is purified in a further distillation step K3 (purifying column) (e.g. (O)), wherein the tops fraction obtained (e.g. (25a)) is a pure alkyl methacrylate product having a content of methacrylonitrile in the range from 10 to 300 ppm, preferably 10 to 100 ppm, more preferably 10 to 80 ppm, especially preferably 50 to 80 ppm, based on the pure alkyl methacrylate product.

The crude alkyl methacrylate product (e.g. (22b)) from distillation step K2 is preferably guided into distillation step K3 (purifying column) in liquid form just below the boiling point of the composition. The feed from distillation step K3 (e.g. (22b)) is preferably in the middle of purifying column K3. The energy input into distillation column K3 is typically effected by means of an evaporator heated with low-pressure steam. Distillation step K3 (purifying column, e.g. (O)), like distillation step K2, is preferably conducted under reduced pressure.

Typically, the distillate stream fully condensed at the top of column K3 (e.g. (O)) is divided into a product stream (e.g. (25a)) and a recycle stream into the column. The quality of the pure alkyl methacrylate product (e.g. (25a)) can be controlled, for example, via the reflux ratio. The bottom stream (25b) is preferably recycled into the esterification (e.g. (E)) or goes directly or indirectly back to the azeotrope column, either as feed stream or having been fed into the condensate from the azeotrope column, in which case proportions can be used as column reflux.

Typically, the bottoms fraction from distillation step K3 (purifying column) (e.g. (O)) can be recycled fully or partly into the third reaction stage (esterification). More particularly, it is possible thereby to recover alkyl methacrylate present.

Variant A

In a preferred embodiment of the invention (also referred to as variant A), the separation of alkyl methacrylate from the third reaction mixture comprises
(i) first distilling the third reaction mixture obtained in the third reaction stage (esterification) in a first distillation step K1 (primary column) (e.g. (F)) to obtain a first water-containing heteroazeotrope (e.g. (14a)) comprising methacrylonitrile and acetone as tops fraction;
(ii) separating the first water-containing heteroazeotrope as condensate in a phase separation step (phase separator i) (e.g. (G)) into an aqueous phase WP-1 (e.g. (15b)) and an organic phase OP-1 (e.g. (15a)) comprising the predominant portion of the alkyl methacrylate;
(iii) guiding the organic phase OP-1 (e.g. (15a)), optionally after an extraction step, into a second distillation step K2 (azeotrope column) (e.g. (L)), wherein the tops fraction obtained is a second water-containing heteroazeotrope (e.g. (22a)) comprising methacrylonitrile and acetone;
(iv) separating at least a portion (e.g. (23a)) of the condensed second water-containing heteroazeotrope (e.g. (22a)) in a phase separation step (phase separator II) (e.g. N) into an aqueous phase WP-2 (e.g. (24b)) comprising methacrylonitrile and acetone, and an organic phase OP-2 (e.g. (24a)), wherein this phase separation is especially promoted by additional addition of water (e.g. (16c)),
   wherein the organic phase OP-2 (e.g. (24a)) is recycled fully or partly into the second distillation step K2,
   and wherein the aqueous phase WP-2 (e.g. (24b)) comprising methacrylonitrile and acetone is partly recycled into the third reaction stage (esterification) and partly discharged from the process, optionally after an extraction step (e.g. (P)).
(iv) preferably comprises separating the condensed water-containing heteroazeotrope (e.g. (23a)) in a phase separation step (phase separator II) (e.g. N) into an aqueous phase WP-2 (e.g. (24b)) comprising methacrylonitrile and acetone, and an organic phase OP-2 (e.g. (24a)), wherein this phase separation is promoted by additional addition of water (e.g. (16c)).

In a preferred embodiment (variant A), the aqueous phase WP-1 (e.g. (15b)) is recycled fully or partly into the third reaction stage (esterification), and the organic phase OP-1 (e.g. (15a)) comprising the predominant portion of the alkyl methacrylate is subjected to an extraction (e.g. (H)) using water as extractant, wherein the aqueous phase of this extraction (e.g. (17b)) is recycled into the third reaction stage and the organic phase (e.g. (17a)) of this extraction is guided into the second distillation step K2 (azeotrope column, e.g. (L)).

Preference is given to adding water, typically demineralized water or well water, in the phase separation step (phase separator II) (e.g. N), in which at least a portion (e.g. (23a)) of the second water-containing heteroazeotrope (e.g. (22a)) is separated into an aqueous phase WP-2 and an organic phase OP-2, which typically improves the phase separation.

In a preferred embodiment (variant A), a portion (e.g. (26b)) of the aqueous phase WP-2 (e.g. (24b)) comprising methacrylonitrile and acetone is subjected to an extraction (e.g. (P)) to obtain an aqueous phase WP-3 (e.g. (28b)) and an organic phase OP-3 (e.g. (28a)), wherein the aqueous phase WP-3 is discharged fully or partly from the process, and wherein the organic phase OP-3 is recycled fully or partly into the third reaction stage. It is optionally possible to at least partly discharge the organic phase OP-3 from the process. The organic phase OP-3 is preferably discharged from the process as cleavage acid (e.g. (27)) together with the waste acid from the esterification (e.g. (11)). More particularly, the aqueous phase WP-3, for example together with the waste acid from the esterification (e.g. (11)), can be sent to a downstream process for regeneration of sulfuric acid or a downstream process for obtaining ammonium sulfate (e.g. via (27)).

Typically, in the above-described variant A, the discharge of troublesome by-products, typically MAN and acetone, is effected via a portion (e.g. (26b)) of the aqueous phase WP-2 (e.g. (24b)), wherein the loss of alkyl methacrylate can be reduced by a downstream extraction step (e.g. (P)).

Preferably, the tops fraction from distillation step K2 (second water-containing heteroazeotrope (e.g. (22a)) is first supplied as a vapour stream to a condenser (e.g. (M)) and condensed stepwise under reduced pressure. What is preferably obtained here is a biphasic condensate I (e.g. (23a)) in the first stage of the condensation (on the suction side of the condenser), which is guided into a phase separator (e.g. (N)). A further condensate II (e.g. 23 (d)) is preferably additionally obtained in the second stage of the condensation (on the pressure side of the condenser), which is used as extractant in the extraction (e.g. (P)) of the aqueous phase WP-2 (e.g. (24b) or a portion of the aqueous phase WP-2 (e.g. (26b)).

In a further embodiment, a portion (e.g. (26b)) of the aqueous phase WP-2 comprising methacrylonitrile and acetone is subjected to an extraction (e.g. (P)) to obtain an aqueous phase WP-3 (e.g. (28b)) and an organic phase OP-3 (e.g. (28a)), wherein the aqueous phase WP-3 is subjected to a further distillation step K5, wherein a tops fraction comprising methacrylonitrile is obtained in distillation step K5, which is discharged from the process, and wherein a bottoms fraction comprising water is obtained in distillation step K5, which is recycled fully or partly into the extraction (e.g. (P)), and wherein the organic phase OP-3 is recycled fully or partly into the third reaction stage. Typically, the bottoms fraction from distillation step K5 is largely free of methacrylonitrile. Typically, with the aid of the further distillation step K5, the discharged wastewater stream (e.g. 28b) can be purified, and the disposal of the waste stream simplified.

Variant B

In a preferred embodiment of the invention (also referred to as variant B), the separation of alkyl methacrylate from the third reaction mixture comprises
(i) first distilling the third reaction mixture obtained in the third reaction stage in a first distillation step K1 (primary column) (e.g. (F)) to obtain a first water-containing heteroazeotrope (e.g. 14b) comprising methacrylonitrile and acetone as tops fraction;
(ii) guiding the first water-containing heteroazeotrope as a vapour stream into a further distillation step K4 (primary stripper, e.g. (1)) in which a further water-containing heteroazeotrope comprising methacrylonitrile and acetone is obtained as tops fraction (e.g. (19a)), and a bottoms fraction (e.g. (19b)) comprising alkyl methacrylate,
(iii) discharging the tops fraction (e.g. (19a)) from distillation step K4, optionally after a scrubbing step (e.g. (J)), fully or partly from the process in the form of a gaseous stream (e.g. (21a));
(iv) separating the bottoms fraction (e.g. (19b)) from distillation step K4 in a phase separation step (phase separator II, e.g. (K)) into an aqueous phase WP-2 (e.g. (20b)) comprising methacrylonitrile and acetone, and an organic phase OP-2 (e.g. (20a)), wherein the aqueous phase WP-2 comprising methacrylonitrile and acetone is recycled fully or partly into the third reaction stage,
(v) guiding the organic phase OP-2 fully or partly into a second distillation step K2 (azeotrope column, e.g. (L)) in which the tops fraction obtained is a second water-containing heteroazeotrope comprising methacrylonitrile and acetone, which is condensed fully or partly (e.g. in (M)) and guided into the phase separation step (phase separator II, e.g. K) according to (iv) (e.g. (23c)).

Typically, in distillation step K4 (primary stripper. e.g. (1)), the tops fraction obtained (e.g. (19a)) is a low-boiling mixture comprising methanol, acetone, methacrylic esters and water, and the bottoms fraction obtained (19b) is an azeotropically boiling mixture comprising alkyl methacrylate and water.

In a preferred embodiment (variant B), the reflux in distillation step K4 (primary stripper) (e.g. (1)) is produced by means of a partial condenser adjusted such that the tops fraction (e.g. (19a)) is discharged from column K4 in the form of a vapour and a liquid condensate comprising alkyl methacrylate is returned to the column as reflux. A portion of the reflux from distillation column K4 is preferably removed in the form of a liquid side stream (e.g. (19c)) and guided as reflux into distillation column K1 (primary column, e.g. (F)).

Typically, the bottoms fraction (e.g. (19b)) from distillation step K4 (primary stripper, e.g. (1)) is an azeotropic mixture comprising alkyl methacrylate, water, small amounts of low boilers (e.g. methanol, acetone) and high boilers (e.g. hydroxyisobutyric esters). The bottoms fraction from distillation step K4 is preferably cooled and separated in a phase separator II (e.g. (K)), preferably together with a further reflux stream (e.g. (23c)) into an organic phase OP-2 (e.g. (20a)) and an aqueous phase WP-2 (e.g. (20b)). Typically, the aqueous phase WP-2 comprises water, alcohol, acetone and alkyl methacrylate. The aqueous phase WP-2 (e.g. (20b)) can preferably be mixed with fresh water, e.g. demineralized water (DM water) (e.g. (16b)), and sent to the esterification (e.g. (E)) in the form of a combined reflux stream (e.g. (20c)). Typically, it is possible thereby to cover the water demand of the esterification and recover reactants.

The tops fraction (e.g. (19a)) from distillation step K4 is preferably guided as a vapour stream into an offgas scrubbing column (e.g. (J)), where it is scrubbed with fresh alcohol (e.g. (10b)), e.g. methanol, as scrubbing medium. The scrubbed offgas stream (e.g. (21a)) is preferably discharged fully or partly from the process. The organic stream (e.g. (21b)) comprising methanol and alkyl methacrylate is preferably obtained in the bottoms from the offgas scrubbing column (e.g. (J)), and is recycled into the esterification (E). This organic reflux stream may be distributed here between various esterification reactors.

Further Steps

In a preferred embodiment, the process according to the invention comprises a regeneration of sulfuric acid, wherein a portion of the third reaction mixture obtained in the third reaction stage and at least one aqueous or organic waste stream comprising sulfuric acid, ammonium hydrogensulfate and sulfonated acetone derivatives that results from the discharge of the water-containing heteroazeotrope comprising methacrylonitrile and acetone is sent to a thermal regeneration step in which sulfuric acid is obtained, which is recycled into the first reaction stage. This process proceeds at temperatures above 900° C. in the gas phase and comprises the thermal cracking of the hydrogensulfate salts, which are oxidized here to nitrogen.

In a preferred embodiment, the process according to the invention comprises obtaining ammonium sulfate, wherein a portion of the third reaction mixture obtained in the third reaction stage and at least one aqueous or organic waste stream comprising sulfuric acid, ammonium hydrogensulfate and sulfonated acetone derivatives that results from the discharge of the water-containing heteroazeotrope comprising methacrylonitrile and acetone is sent to a thermal regeneration step in which ammonium sulfate is obtained by means of crystallization, which is separated off as a by-product. Neutralization is typically necessary here, which is effected by addition of aqueous ammonia or ammonia itself. The workup of the waste acid by means of what is called wet oxidation in the presence of homogeneous catalysts (e.g. copper sulfate) is typically a further means of processing.

A waste stream (e.g. (11)) consisting essentially of dilute sulfuric acid which is removed from the reactor III for esterification and/or one or more waste streams from the process (e.g. (28b) or (26a)) is preferably sent to a process for regeneration of sulfuric acid or to a process for obtaining ammonium sulfate. Preference is given to supplying the cleavage acid (27) according to FIGS. 1-3 to a process for regeneration of sulfuric acid or a process for obtaining ammonium sulfate.

Processes for regeneration of sulfuric acid and processes for obtaining ammonium sulfate from cleavage acid are known to the person skilled in the art and are described, for example, in WO 02/23088 A1 and WO 02/23089 A1. The embedding of processes for regeneration of sulfuric acid into a process for preparing alkyl methacrylates by the ACH-sulfo process is described, for example, in DE 10 2006 059 513 or DE 10 2006 058 250.

It is further preferable to likewise supply the offgases obtained from the reaction stages of amidation and conversion to the thermal sulfuric acid regeneration step.

### DESCRIPTION OF THE FIGURES

FIG. 1 shows a flow diagram of preferred embodiments of the process according to the invention. FIG. 1 shows the preferred elements of an integrated plant for continuous preparation and purification of alkyl methacrylates, especially methyl methacrylate (MMA). The integrated plant shown has various plants connected to one another, usually in a fluid-conducting manner, as elements of this integrated system. This integrated plant includes the preparation of methacrylamide or the sulfuric acid solution thereof, consisting of the process steps of amidation (A, B) and conversion (C, D), followed by an esterification (E), followed by a workup of the reaction product (F, G, H, I, J, K), followed in turn by a fine purification (L, M, N, O). Solid lines preferentially describe the flow pathways of the process according to variant A; dotted lines preferentially describe the flow pathways of the alternative process according to variant B. A combination of apparatuses and streams of matter from the two variants is likewise possible.

FIG. 2 shows a schematic flow diagram of a first preferred embodiment of the process according to the invention (variant A).

FIG. 3 shows a schematic flow diagram of a second preferred embodiment of the process according to the invention (variant B).

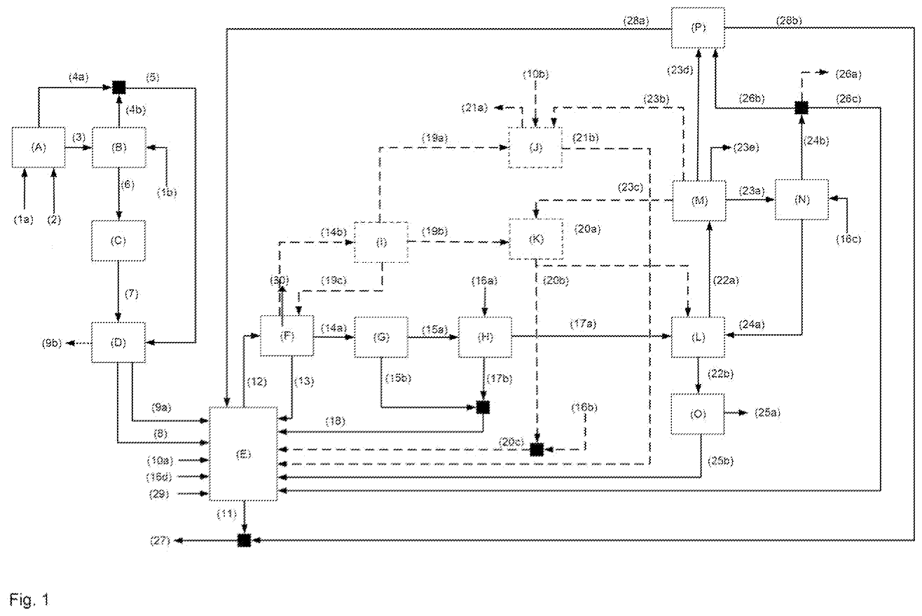

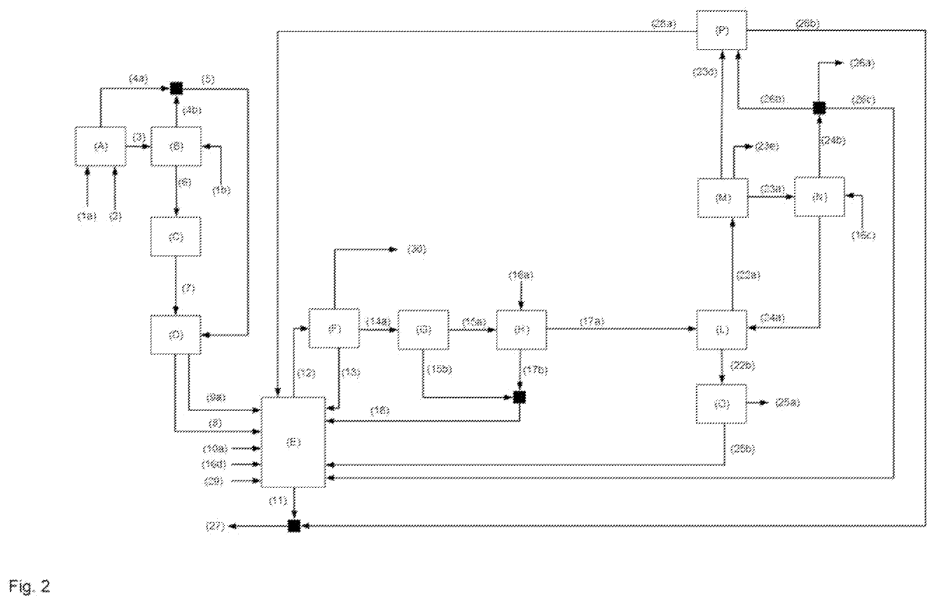

In FIGS. 1 to 3, the reference symbols have the following meanings:

Apparatuses
(A) Stage 1 amidation reactor
(B) Stage 2 amidation reactor
(C) Heater
(D) Gas separator/intermediate vessel
(E) Esterification reactor/cascade
(F) Primary column (column K1)
(G) Phase separator I
(H) Scrubbing column (extraction I)
(I) Primary stripper column (column K4)
(J) Offgas scrubbing column
(K) Phase separator for crude MMA
(L) Azeotrope column (column K2)
(M) Condenser/vacuum system
(N) Phase separator II
(O) Purifying column (column K3)
(P) Extraction column II (extraction of pump condensate)

Streams of Matter
(1a) Acetone cyanohydrin feed to stage 1
(1b) Acetone cyanohydrin feed to stage 2
(2) Sulfuric acid feed
(3) Amide mixture exiting stage 1
(4a) Offgas from stage 1 amidation reactors
(4b) Offgas from stage 2 amidation reactors
(5a) Optional offgas from stage 1 & 2 amidation reactors
(5b) Offgas from stage 1 & stage 2 amidation reactors
(6) Amide mixture exiting stage 2
(7) Converted amide mixture
(8) Degassed amide mixture
(9a) Gas separator offgas for esterification
(9b) Optional offgas, removed from the process
(10a) Alcohol feed (for MMA: methanol)
(10b) Alcohol feed to offgas scrubbing column
(11) Cleavage acid from esterification
(12) Vapour stream from esterification
(13) Liquid reflux stream from primary column
(14a) Distillate stream from primary column
(14b) Vapour stream from primary column
(15a) Organic phase from phase separator I (OP-1)
(15b) Aqueous phase from phase separator I (WP-1)
(16a) Demineralized water feed to extraction
(16b) Demineralized water feed
(16c) Demineralized water feed to phase separator II (N)
(16d) Direct steam
(17a) Washed organic phase (OP-1)
(17b) Aqueous phase from extraction
(18) Combined aqueous phases
(19a) Vapour stream from primary stripper column
(19b) Bottom stream from primary stripper column
(19c) Organic sidestream from primary stripper column/reflux from primary column
(20a) Organic phase from phase separation I (OP-1)
(20b) Aqueous phase from phase separation I (WP-1)
(20c) Combined reflux stream/product water
(21a) Offgas from offgas scrubbing column
(21b) Bottom stream from offgas scrubbing column
(22a) Vapour from azeotrope column
(22b) Bottom product from azeotrope column/crude alkyl methacrylate product
(23a) Condensate I to phase separator II
(23b) Offgas from azeotrope column/vacuum system
(23c) Circulation stream condensate
(23d) Condensate II, vacuum pump condensate
(23e) Offgas/inert content of the condensation/vacuum system
(24a) Organic phase from phase separator II (OP-2)
(24b) Aqueous phase from phase separator II (WP-2)
(25a) Top product from purifying column/pure alkyl methacrylate product
(25b) Bottom product from purifying column
(26a) Discharge of aqueous phase from phase separator II
(26b) Aqueous phase to pump condensate extraction
(26c) Aqueous phase to esterification (recycle)
(27) Cleavage acid
(28a) Organic phase from extraction of pump condensate (OP-3)
(28b) Aqueous phase from extraction of pump condensate (WP-3)/raffinate to cleavage acid
(29) Methanol/methyl methacrylate mixture
(30) Vacuum pump condensate

FIG. 4 describes the reaction network of the formation of methacrylic acid and/or methyl methacrylate proceeding from methane and ammonia, and acetone. Proceeding from methane (CH₄) and ammonia (NH₃), it is possible to prepare hydrogen cyanide via the BMA process (hydrogen cyanide from methane and ammonia) by means of catalytic dehydrogenation (CH₄+NH₃→HCN+3H₂) (variant 1). Alternatively, it is possible to prepare hydrogen cyanide via the Andrussow process proceeding from methane and ammonia, with addition of oxygen (CH₄+NH₃+1.5 O₂→HCN+3H₂O) (variant 2). In the next step, proceeding from acetone and hydrogen cyanide, acetone cyanohydrin (ACH) is prepared with addition of a basic catalyst (e.g. diethylamine Et₂NH or else alkali metal hydroxides). The hydroxyl group of acetone cyanohydrin is subsequently esterified with sulfuric acid, initially giving sulfoxyisobutyronitrile (SIBN). The nitrile group of sulfoxyisobutyronitrile (SIBN) can be hydrolysed in the next step under the action of sulfuric acid and water, giving sulfoxyisobutyramide hydrogensulfate (SIBA·H₂SO₄). A side reaction that can proceed is the formation of methacrylonitrile (MAN) with elimination of sulfuric acid from SIBN. Sulfoxyisobutyramide hydrogensulfate (SIBA·H₂SO₄) can additionally be partly hydrolysed to give alpha-hydroxyisobutyramide hydrogensulfate (HIBAm·H₂SO₄). Likewise possible is the reverse reaction to give the sulfuric ester SIBA·H₂SO₄. A by-product formed may be alpha-hydroxyisobutyric acid (HIBAc) via further hydrolysis of HIBAm·H₂SO₄. Proceeding from SIBA·H₂SO₄, with the elimination of sulfuric acid, methacrylamide hydrogensulfate (MAA·H₂SO₄) is formed (conversion). The gradual reaction of HIBAm or HIBAc to give MA or MAA can likewise proceed as an elimination reaction with elimination of NH₄HSO₄ or water. Methacrylamide hydrogensulfate (MAA·H₂SO₄) can subsequently be converted by hydrolysis to methacrylic acid (MA) or by esterification with methanol (MeOH) methyl methacrylate (MMA). If alpha-hydroxyisobutyric acid (HIBAc) is introduced into the esterification, it can be converted to methyl alpha-hydroxyisobutyrate (MHIB).

The abbreviations in FIG. 4 have the following meanings:
ACH acetone cyanohydrin;
SIBN alpha-sulfoxyisobutyronitrile;
SIBA alpha-sulfoxyisobutyramide;
SIBA·H₂SO₄ alpha-sulfoxyisobutyramide hydrogensulfate;
MAN methacrylonitrile;
HIBA alpha-hydroxyisobutyramide;
HIBAm·H₂SO₄ alpha-hydroxyisobutyramide hydrogensulfate;
MAA methacrylamide;
MAA·H₂SO₄ methacrylamide hydrogensulfate;
MA methacrylic acid;
MMA methyl methacrylate;
HIBAc alpha-hydroxyisobutyric acid;
MHIB methyl alpha-hydroxyisobutyrate

Embodiment of the Process According to FIG. 2 (Variant A)

One possible embodiment of the process (variant A) relating to the preparation of alkyl methacrylate, especially MMA, according to the flow diagram in FIG. 2 is described hereinafter:

In the amidation reactors (A) and (B), which take the form of a loop reactor, ACH and sulfuric acid are converted to a sulfuric acid solution comprising SIBA, HIBAm and MAA (each predominantly in the form of the hydrogensulfates). Depending on the reaction conditions, especially in reactors (A) and (B), MAN may be formed as a by-product from ACH with release of water. The loop reactors (A) and (B) each comprise the following elements: circulation pump, static mixer, heat exchanger and gas separator.

The amidation reactor (A) of stage 1 has an ACH feed (1a) and a sulfuric acid feed (2). The ACH feed (1a) opens into the circuit of the loop reactor (A) on the pressure side of the circulation pump, but upstream of the static mixer. The sulfuric acid feed (2) opens into the circuit of the loop reactor (A) upstream of the ACH feed (1a) and on the suction side of the circulation pump, which can preferably improve the pumpability of the gas-containing reaction mixture.

The reaction mixture in loop reactor (A) is pumped in circulation within the temperature range of 70-130° C. and at a circulation ratio (ratio of circulation volume flow rate to feed volume flow rate) in the range from 5 to 110, and the temperature can be adjusted by means of secondary water-cooled shell-and-tube heat exchangers. More particularly, the heat of reaction of the strongly exothermic reaction between acetone cyanohydrin and sulfuric acid is removed. The static dwell time in the reactor circuit of the amidation reactor (A) is in the range from 5 to 35 minutes. The amidation reactor (A) is operated at standard pressure. The blended and temperature-controlled reaction mixture is then introduced into a gas separator. The selective separation of gaseous secondary components (such as carbon monoxide and other inerts/low boilers) from the amide circulation stream and the discharge of the offgas stream (4a) are effected here.

A substream (3) of the reaction mixture pumped in circulation is fed to the second loop reactor (B) by means of a discharge pump, by gravimetric means or with supply pressure from the reactor circulation pump itself, and heated up by an additional heat exchanger if required. For further conversion of the reaction mixture (3), the amidation reactor (B) is supplied with fresh acetone cyanohydrin via the ACH feed (1b). Loop reactor (B) is configured in a comparable manner to loop reactor (A) in terms of temperature, pressure, dwell time and flow pathway.

Gaseous by-products are removed from reactor (B) in the form of the offgas stream (4b).

The resultant offgas streams (4a, 4b) are combined by means of interconnection to give (5) and sent to the downstream gas separator/intermediate vessel (D) for the purpose of utilization. Alternatively, the reaction offgases (4a, 4b) are removed from the process as a combined offgas stream (5a).

The resultant liquid reaction mixture (6) is subjected to a conversion (C) for maximum conversion to MAA. The conversion is typically composed of one or more heat exchangers, with controlled heating and subsequent dwell time of the entering reaction mixture (6) maximizing the concentration of MAA in the product stream exiting from the amidation, in the converted amide mixture (7).

The converted amide mixture (7) is sent gravimetrically, for example, to the gas separator/intermediate vessel (D). The resultant offgas is separated here from the viscous and hot converted amide mixture (7). The offgas released comprises mainly carbon monoxide that forms through breakdown reactions, and additionally ultrafine droplets of methacrylamide-containing reaction mixture. The reactant-containing overall offgas (9a) from the gas separator/intermediate vessel (D) is therefore passed onward into the esterification (E). The degassed amide mixture (8) is subsequently pumped or fed gravimetrically to the esterification (E).

The offgas stream (5b) from the amidation stages can be connected on the gas side to the gas separation vessel (D), and the overall offgas (9a) from process steps (A, B, C, D) may be connected to the vapour space of the esterification (E). Alternatively, the offgas from (D) can at least partly be removed from the process as offgas stream (9b).

In the esterification (E), the reactants required for conversion of methacrylamide to the corresponding ester are fed in directly or indirectly in the form of the corresponding alcohol (10a, 10b) and of demineralized water (16a, 16b, 16c). The degassed amide mixture (8) is fed to the reaction (E) here through introduction tubes or immersed tubes, in a pumped or gravimetric manner. A direct alcohol feed (10a) (e.g. methanol for the preparation of MMA) is usually effected by means of immersed introduction tubes or static mixers in the feed to the esterification (E).

In addition, various circulation streams from the thermal workup (F, G, H, L, M, N, O, P) are connected to the esterification reactor (E) as shown in FIG. 2.

The esterification is typically conducted in one or more esterification reactors (E) that are mixed by means of a stirrer or pump and are gravimetrically connected to one another. A further form of mixing is convection, which is caused by the supply of evaporable reactants. The esterification reactors are often equipped with heat exchangers in order to assure the input of heat for the esterification reactors. For example, the heat input is achieved by jacket heating, forced circulation evaporator or direct feeding of steam.

The reaction mixture (crude ester) formed in the esterification (E) is guided out of the esterification reactor (E) by distillation as a continuous vapour stream (12). The vapour stream (12) may also be combined here from multiple reactors (E). The acid mixture (11) remaining in the esterification reactors, after intensive distillative removal of residual product, is discharged from the esterification.

The vapour stream from the esterification (12) is subjected to a counter current distillation in the primary column (F). The vapour stream may be condensed at the top of the column (F) as reflux from the primary column (F) and partly returned. The offgas (30) obtained beyond the condensation, which is generated by the supply of stream (9a) inter alia, can be removed from the process and sent to incineration, for example.

The bottom product (13) comprising MAA is returned continuously to the esterification reactor (E). It is possible to distribute the bottom product (13) between multiple esterification tanks of the esterification (E).

The vapour stream (14a) at the top of the column (F) contains the predominant proportion of the alkyl methacrylate, and also water, alcohol, acetone and MAN. Methacrylic acid forms a low-boiling azeotrope with water and is likewise present in the vapour stream (12).

The aqueous and condensed vapour stream (14a) at the top of the column (F) is subjected to a phase separation (G) in the phase separator I, in which an organic phase (15a) comprising alkyl methacrylate, methanol, acetone and MAN, and an aqueous phase (15b) are obtained.

The organic phase (15a) is subjected to a liquid/liquid extraction (H), especially in order to return a large portion of the methanol present to the esterification (E). For this purpose, the organic phase (15a) is extracted in a stirred extraction column (H) with demineralized water (16a) in countercurrent. The resultant aqueous phase (17b) is combined with the aqueous phase (15b) from the phase separator (G) in stream (18) and returned to the esterification (E). The organic phase (17a) which is present in extraction step (H) and comprises the predominant portion of alkyl methacrylate and significant proportions of low and high boilers is sent to further thermal workup (L, M, N, O).

The organic phase (17a) from extraction step (H) is subjected to an azeotropic distillation (L) under reduced pressure in a further step. The azeotrope column is implemented in the form of a stripping column, wherein the organic feed (17a) is guided preheated to the top of the column (L), which is heated indirectly with low-pressure steam by an evaporator. At the top of the column (L), a heteroazeotropic mixture (22a) comprising MMA, water, methanol, acetone, MAN and further low boilers is obtained. The bottom product (22b) separated off is purified alkyl methacrylate (crude alkyl methacrylate). The vapour stream (22a) leaves the column (L) in vaporous form and is condensed stepwise under reduced pressure in the downstream condenser (M).

The main condensation in (M) proceeds on the suction side of the vacuum unit, forming a liquid condensate (23a) which is subjected to a phase separation in the phase separator II (N). On the pressure side of the condensation unit (M), a further liquid stream (23d, vacuum pump condensate) is generated, which serves as extractant in the extraction step (P). The inert gas-containing offgas (23e) formed in the condensation on the pressure side is removed from the process.

The liquid condensate (23a) from (M) is guided into the phase separation (N) with addition of demineralized water (16c) and separated into an organic phase (24a) and an aqueous phase (24b). The organic phase (24a) contains a certain proportion of alkyl methacrylate and is guided back into the distillation step (L) via the top of the column (L).

The aqueous phase (24b) from (N), in a corresponding manner to the added fresh water (16c), is saturated with water-soluble components such as methanol, acetone and MAN, and is divided into two streams for avoidance of by-product enrichment. A substream (26c) is returned to the esterification reactor (E) in the form of a circulation stream. A substream (26b) is discharged from the process via (28b) after an extraction step (P).

As an alternative to stream (26b, 26c), it is likewise possible to discard stream (24b) completely in the form of stream (26a) and discharge it from the process.

Stream (26b) serves as an outlet for enriched secondary components, which, for the purpose of recovery of alkyl methacrylate, is sent to an extraction column (D) (PK extraction column). In the extraction step (P), the condensate (23d) from condenser (M) is used as extractant, with guiding of the streams (23d) and (26b) in countercurrent. In the extraction step (B), an aqueous phase (28b) and an organic phase (28a) are obtained, wherein the aqueous phase (28b) is mixed with the waste acid (11) and discharged fully from the process as cleavage acid (27), and wherein the organic phase (28a) is fed into the esterification (E) as stream of value comprising alkyl methacrylate.

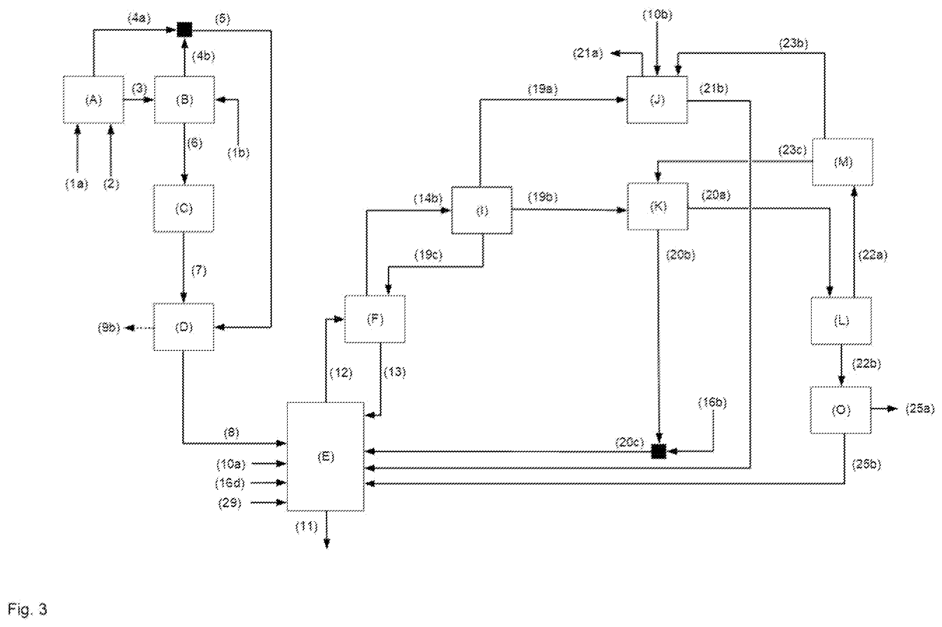

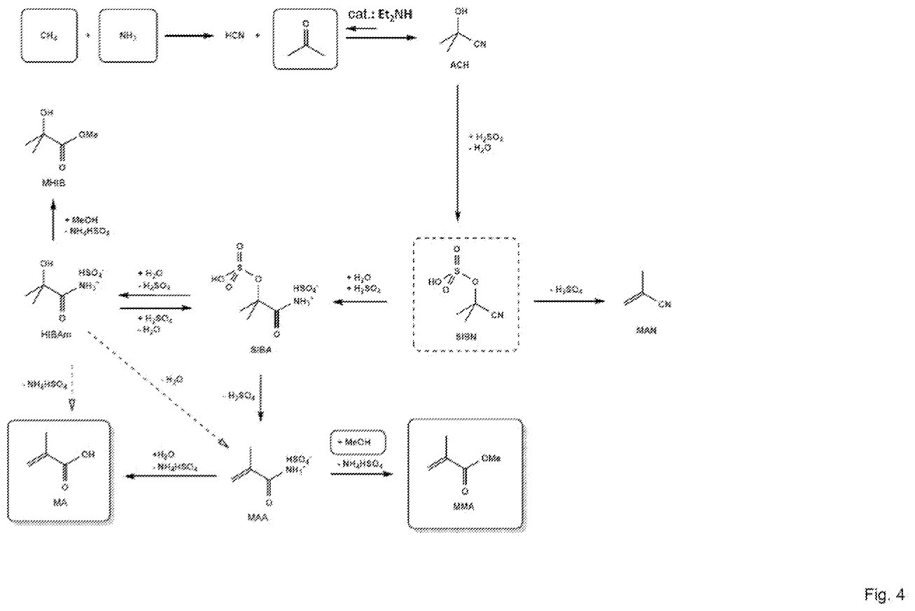

Embodiment of the Process According to FIG. 3 (Variant B)

One possible embodiment of the process (variant B) relating to the preparation of alkyl methacrylate, especially MMA, according to the flow diagram in FIG. 3 is described hereinafter:

The amidation in (A) and (B), the conversion in (C), the gas separation in (D) and the esterification in (E) are effected as described in the embodiment according to FIG. 2 (variant A). In addition, various circulation streams from the thermal workup (F, I, J, K, L, M, O) are connected to the esterification reactor (E) as shown in FIG. 3.

In the embodiment according to variant B (FIG. 3), the vapour stream formed in the primary column (F) is fed uncondensed as a vapour stream (14b) to a further distillation step (1) (primary stripper column). In the primary stripper column (I), the tops fraction obtained (19a) is a low-boiling mixture comprising methanol, acetone, methacrylonitrile, methacrylic esters and water, and the bottoms fraction obtained (19b) is an azeotropically boiling mixture crude ester/water mixture. The reflux for column (I) is generated by means of a partial condenser which is adjusted such that the low-boiling mixture (19a) of methanol, acetone, alkyl methacrylate and water is discharged in the form of a vapour, while an alkyl methacrylate-rich mixture can be returned to the column.

A portion of the liquid descending within the primary stripper column (I) is drawn off in the upper region of the column (I) in the form of a liquid side stream (19c) and used as reflux for the primary column (F).

The azeotropic mixture (19b) drawn off in the bottoms from the primary stripper column (I), comprising methacrylate esters, water, small amounts of low boilers (e.g. methanol, acetone) and high boilers (e.g. hydroxyisobutyric esters), is cooled down and sent to the phase separator II (K). The bottom product (19b) is separated into an organic phase (20a) and an aqueous phase (20b) in the phase separator (K). In addition, in the phase separator (K), the reflux stream (23c) that results from the condenser (M) and the top product from the azeotrope column (L) is separated into an organic phase (20a) and an aqueous phase (20b).

The aqueous phase (20b) from the phase separation (K) comprising water, saturated with methanol, acetone and methacrylic esters, is mixed with demineralized water (16b) and fed to the esterification (E) in the form of a combined reflux stream (20c). The reflux stream (20c) may especially serve to cover the water demand of the esterification and recover reactants.

The vapour stream (19a) that leaves the primary stripper column (I) is guided into a scrubbing step (J) (offgas scrubbing column) and scrubbed with fresh alcohol (10b), e.g. methanol, as scrubbing medium, which largely frees the gas stream from alkyl methacrylate.

The offgas scrubbing column (J) is operated in countercurrent. Alkyl methacrylate-containing output air (23b) from condenser (M) is likewise fed to the offgas scrubbing column (J) in order to recover alkyl methacrylate.

The organic stream (21b) comprising methanol and alkyl methacrylate is obtained in the bottoms from the offgas scrubbing column (J), and is recycled into the esterification (E). The organic reflux stream (21b) may be distributed here between various esterification reactors.

At the top of the offgas scrubbing column (J), an offgas stream (21a) is obtained, including MAN, dimethyl ether and methyl formate, and also saturation concentrations of methanol and acetone, and containing little alkyl methacrylate. By adjusting the ratio of methanol (10b) to vapour (19a) and the top temperature in (J), it is possible to vary the composition of the offgas stream (21a). The offgas stream (19a) comprising methanol, acetone and MAN is discharged fully from the process.

The organic phase (20a) from the phase separation (K) is guided into the azeotrope column (L). The organic phase (20a) comprising the predominant proportion of alkyl methacrylate and significant proportions of low and high boilers is sent to further thermal workup (L, M, N, O).

The thermal workup (L, M, N, O) is effected essentially as described above in the embodiment according to variant A (FIG. 2). By contrast with variant A, streams (23b) and (23c) formed in the condenser (M) are returned to the process steps (J) and (K). A fully condensed and azeotropic low boiler mixture (23c) which is obtained from the tops fraction from the azeotropic column (L) via condenser (M), and which typically has two liquid phases, is guided into the phase separation (K) in the upstream step. Stream (23c) is composed of streams (23a) and (23d) from variant A. The offgas (23b) formed in the condensation/vacuum unit (M) is guided into the scrubbing column (J), wherein materials of value in particular, such as alkyl methacrylate, are recovered.

The invention is described further by the examples that follow. Example A1 (comparative example) demonstrates the operation of a process for preparing methyl methacrylate with a noninventive sulfuric acid concentration (100.3% by weight) and a low discharge of the methacrylonitrile-containing aqueous phase from a phase separator II, combined with a low overall MMA yields to obtain an MMA product having a high content of the MAN by-product.

Inventive example A2 describes the preparation of methyl methacrylate having the claimed features, with achievement of a high overall yield of MMA, characterized by reduced MAN formation in the amidation and conversion, and by a moderation of the steady-state MAN concentration in the workup section via controlled discharge, combined with the achievement of a low MAN content in the MMA target product.

Example A3 (comparative example) illustrates operation analogously to comparative example 1, but additionally without extraction of the aqueous MAN-containing stream discharged, which brings another increase in MMA losses.

Beyond that, example B1 (inventive) describes the preparation of methyl methacrylate having the claimed features, with achievement of a high overall yield of MMA, characterized by reduced MAN formation in the amidation and conversion and an exclusively distillative removal of by-products (as vapour).

### EXAMPLES

### Examples A1, A2 and A3 According to FIG. 2 (Variant A)

The preparation of methyl methacrylate comprising the reaction of acetone cyanohydrin with sulfuric acid in the amidation/conversion ((A), (B), (C), (D)), the subsequent esterification (E) with methanol, and distillative and extractive workup ((F), (G), (H), (L), (O), (M), (N), (P)) of the methyl methacrylate product was effected by the embodiment according to FIG. 2 as described above (variant A).

A mass balance and assessment of the discharge of methacrylonitrile (MAN) and acetone via (28b), output air streams (30) and (23e), and via the MMA product (25a) was effected (see flow diagram according to FIG. 2).

Described hereinafter are comparative examples (Examples A1 and A3) using sulfuric acid with a concentration of 100.3% by weight (formally 0.3% by weight of free SO₃) and an inventive example (Example A2) using sulfuric acid with a concentration of 99.7% by weight (0.3% by weight of water).

The water content of the ACH feed streams (1a) and (1b) is calculated from the difference from the ACH content which is ascertained by means of HPLC, or via an analysis by means of gas chromatography (with thermal conductivity detector) which is quantitative and selective specifically for water.

The water content in the sulfuric acid feed (2) is calculated from the difference from the sulfuric acid content which is ascertained by measuring the density and speed of sound.

The general procedure for the process according to FIG. 2 (variant A) is described hereinafter, with differences and results shown in Tables 1 to 3.

General Process Procedure:

1a. Reaction Stages (A), (B), (C), (D) and (E)

5000 kg/h of acetone cyanohydrin having a composition of 98.8% by weight of acetone cyanohydrin, 0.25% by weight of acetone, 0.65% by weight by water and free sulfuric acid was divided in a mass ratio of 75/25, so as to obtain a stream (1a) of 3750 kg/h and a stream (1b) of 1250 kg/h. Feed stream (1a) was subsequently applied to the first amidation reactor (A).

The loop reactor (A) was composed of the following elements connected by pipeline: circulation pump, static mixer, heat transferrer, cooler, and a gas separator. A circulation volume flow rate of 350 m³/h was established in the reactor (A), such that effective heat transfer and effective mixing and gas separation were possible. The overall reactor circuit was operated at about 95° C. and 990 mbar(a) at slightly reduced pressure.

Feed stream (1a), continuously and at a temperature of about 20° C., was fed to the reactor circuit (A) described and mixed in.

The amount of sulfuric acid (2) needed for the optimal conversion of the reaction mixture in reactors (A) and (B) that had a concentration according to Table 1 was fed to the reactor (A) in a load-dependent manner in the specified mass ratio to the total amount of ACH (1a+1b). This achieved a sulfuric acid excess (sulfuric acid/ACH ratio of 2.6 kg/kg) in reactor (A).

The resultant stirred-up mixture (3) comprising sulfoxyisobutyramide, methacrylamide and sulfuric acid was then transferred into the second amidation reactor (B), while the offgas (4a) separated off in reactor (A) was sent in the direction of conversion (D).

Reactor (B) was of analogous construction to reactor (A) and was operated under the same physical conditions and parameters. The offgas (4b) formed by side reaction was separated from the reaction mixture by means of a gas separator. The offgases from the amidation (4a) and (4b) were subsequently combined and supplied in the form of offgas stream (5b), the amount of which was about 60 m³/h, to a further gas separator/intermediate vessel (D).

The ACH stream (1b) was subsequently added to the reaction mixture in the second reactor (B). The mass ratio H₂SO₄/ACH established in the reaction mixture is reported in Table 1.

Over the course of the amidation reactors (A, B), a reaction mixture (6) at 95° C. comprising sulfoxyisobutyramide (SIBA), methacrylamide (MAA) and hydroxyisobutyramide (HIBAm), dissolved in the sulfuric acid reaction matrix, was obtained. This mixture was then subjected to a conversion step (C). The reaction mixture was heated therein to 155° C. within a short time and then converted thermally in a delay zone.

After the conversion (C), the methacrylamide-enriched reaction mixture (7) was supplied gravimetrically to a further gas separator or intermediate vessel (D) which was operated at a slightly reduced pressure of about 950 mbara and at a temperature of 155° C.

In the gas separator/intermediate vessel (D), the gas present in the reaction mixture was separated off and combined with offgas (5b). An overall offgas (9a) was obtained, which was supplied to the esterification (E) in gaseous form. The amount and composition of (9a), with regard to the acetone and MAN by-products, are collated in Table 1.

In addition, after the gas separation (D), a stream (8) was obtained, the mass flow rate and composition of which are reported in Table 1. The resultant amount of convertible reactants (MAA+MA) that are guided into the esterification and the amidation yield (MAA+MA) based on ACH are shown in Table 1.

The methacrylamide-containing stream (8), as well as the main components mentioned, contains acetone and MAN as important by-products in the amounts specified in Table 1. The overall mass flow rates of acetone and MAN that are fed to the esterification (E) via (8) and the gaseous feed stream (9a) are reported in Table 1.

The liquid reaction mixture (8) at more than 150° C. was fed to the esterification (E), wherein the feeding was effected into a reactor cascade consisting of three jacket-heated reactor tanks having virtually ideal mixing by free efflux by means of an immersed tube. Stream (8) was reacted in process step (E) with a total of 1540 kg/h of methanol (10a), 590 kg/h of MMA/MeOH mixture (29), 500 kg/h of direct steam (16d) and 1700 kg/h of water (feed streams (16a) and (16c)) at about 120±5° C. and a slightly elevated pressure of 50-150 mbarg. The steady-state substance mixture (29) established consisted, on average, of 25% by weight of methyl methacrylate and 75% by weight of methanol, and also water, and was fed to the utilization of methanol as reactant in the esterification reaction and methyl methacrylate as product (E).

Suitable interconnection of the reactant streams (10a), (16a), (16c); (16d), (29) and of the circulation streams (13), (18), (25b), (26c), (28a) in the esterification (E) achieved a local stoichiometric excess of methanol and water based on the methacrylamide and methacrylic acid substances convertible to methyl methacrylate in each of the esterification tanks.

In a side reaction of the esterification, a portion of the amount of MAN supplied was converted to methacrylamide by hydrolysis and hence lost from the process. The higher the concentration of MAN fed in, the higher the level of hydrolysis as well. In this way, the amounts (MAN(hydrol.)) of MAN reported in Table 1 were hydrolysed in (E).

At the exit from the reactor unit (E), a post-evaporation operated with direct steam was connected, which reduces the proportion of monomers in the acid mixture to a content of <0.1% by weight of MMA, <0.1% of MA, <0.1% by weight of MAA, and also MAN and acetone, according to Table 1, while the water content of the effluxing acid mixture was kept virtually constant. There were likewise further nonvolatile organic by-products present in the waste acid (11), which were discharged in solution as TOC (total organic carbon) and to a certain degree also as polymeric solids. On account of side reactions in the esterification (E), the amounts of sulfonated acetone (Sulfo acetone) reported in Table 1 were present in the form of TOC in the process acid (11). The waste acid contained essentially NH₄HSO₄, H₂SO₄ and water. The TOC content of (11) averaged 2-3% by weight. The amount and composition of the waste acid (11) are compiled in Table 1.

The energy input into (E) for continuous evaporation of the products obtained in (E) was effected by means of 10 barg hot steam.

1b. Prepurification (F), (G), (H)

The crude product formed in the esterification reaction and the methyl methacrylate (MMA) introduced via (29) were withdrawn continuously from the esterification cascade (E) in the form of a vapour stream (12). For this purpose, the esterification reactors were connected to a vapour conduit on the steam side, such that the vapour stream (12) was obtained as the cumulative stream from the reactors II. According to the vapour/liquid equilibrium of the reaction mixtures, the vapour stream (12) was a heteroazeotropic composition comprising MMA, water, MeOH, MA, acetone and MAN as reported in Table 2.

In addition to vapour stream (12), the offgas stream from the amidation (9a) was also fed to the bottom of the primary column (F). Vapour stream (12) was subsequently subjected to a countercurrent distillation by adding the vaporous stream (12) and the gaseous stream (9a) in the bottom region of a column (F). At the top of the column (F), full condensation was effected in condensers that were operated by means of cooling water and cold water. The biphasic distillates were combined, and a substream was guided into primary column (F) as reflux.

The offgas (30) obtained beyond the condensation, which was generated by the supply of stream (9a) inter alia, was removed and sent to an incineration. Via (30), MAN and acetone were discharged from the process in the amounts reported in Table 2.

In accordance with the reflux ratio, a liquid distillate stream (14a) and a liquid bottom stream (13) were obtained at the top of the column (F). The bottom stream (13) was recycled continuously into the esterification reaction. The heteroazeotropic distillate stream (14a), for the purpose of liquid/liquid phase separation, was fed to a phase separator (G) in which two product streams were obtained. The organic light phase (15a) comprising MMA, water, methanol, acetone, MA, MAN and high and low boilers was subjected to a liquid/liquid extraction (H).

The aqueous heavy phase (15b) was first combined with the aqueous raffinate phase (17b) to give a reflux stream (18) that included water, methanol, MMA, acetone, MA, MAN and high and low boilers. Stream (18), analogously to stream (13), was recycled continuously into the esterification reaction (E). The amounts and composition of (13), (14a), (15a), (15b) and (18) are collated in Table 2.

In the extraction step (H), stream (15a) was supplied with deionized water (16a) in order to remove further water-soluble components from the present crude MMA. For this purpose, at the top of the disc extraction column (H), deionized water was fed in continuously in countercurrent to stream (15a), such that, at the bottom of the column (H), an aqueous efflux stream (17b) and a prepurified crude MMA (17a) were obtained. The crude MMA stream (17a) contained MMA, water, methanol, acetone, MA, MAN and high and low boilers. The amounts and composition of (17a) and (17b) are collated in Table 2.

1c. Fine Purification (L), (O), (M), (N), (P)

Stream (17a) was then subjected to a distillative purification (L). For removal of high and low boilers, stream (17a) was fed to the top region of an azeotrope column (L) operated under reduced pressure (300 mbara), which was heated indirectly with hot steam. A low boiler-enriched heteroazeotropic vapour stream (22a) was separated from a methyl methacrylate-enriched bottom stream (22b). The vapour stream (22a) contained MMA, water, MAN, acetone and further low boilers. The bottom stream (22b) contained MMA, MA, high boilers. MAN and acetone. The amounts and compositions are collated in Table 3.

At the exit from the column, on the tube side, the vapour stream (22a) was fed to a condensation/vacuum unit (M) that first subjected the vapour stream (22a) to a main condensation on the vacuum side, then compressed the residual gas in a vacuum pump and again subjected it to postcondensation on the pressure side of the compression process.

The heteroazeotropic distillate (23a) obtained after the main condensation was subjected to a phase separation (N) for further workup, while the pump distillate (23d) obtained on the pressure side was sent to the esterification reaction (E). The pump distillate (23d) contained low boilers, MMA, acetone, methanol, water and MAN. Amounts and compositions are described in Table 3.

The process offgas (23e) obtained beyond the postcondensation on the gas side was discharged continuously from the process. The process offgas (23e) contained low boilers and inert substances that are chemically reactive under given conditions, and also MAN and acetone. Amounts and compositions are described in Table 3.

For improvement of the phase separation, the distillate stream (23a) was supplied with deionized water (16c) in the phase separator (N), such that an organic phase (24a) and an aqueous phase (24b) were obtained. The light organic phase (24a) was circulated here continuously as reflux to the top of the column (L), while the aqueous phase (24b) comprising acetone and MAN was fed to the next process step. Amounts and compositions are described in Table 3.

In Comparative Example A1, stream (24b) was then divided in a fixed mass ratio of 80/20 as stream (26b) and stream (26c). 80% of stream (24b) was recycled directly into the esterification reaction as (26c). 20% of stream (24b) was discharged from the process in the form of stream (28b) via the intermediate step of a liquid/liquid extraction (P) for recovery of MMA. In this way, MAN and acetone were discharged from the process, and hence the enrichment thereof in the process was reduced, monitored and controlled. Amounts and compositions are described in Tables 3 and 4.

In Inventive Example A2, stream (24b) was first divided in a mass ratio of 50/50 into stream (26b) and stream (26c). Stream (26c) was recycled directly into the esterification reaction (E). Stream (26b) was partly discharged from the process via the intermediate step of a liquid/liquid extraction (P) as (28b).

In Comparative Example A3, stream (24b) was divided in a fixed mass ratio of 80/20 as stream (26a) and stream (26c). 80% of stream (24b) was recycled directly into the esterification reaction as (26c). 20% of stream (24b) was removed directly from the process as stream (26a) and not sent to any extraction step (P) for recovery of MMA. In this way, MAN and acetone were discharged from the process. Amounts and compositions are described in Table 3.

Alternatively, stream (24b) can also be removed from the process fully or partly in the form of a discharge stream (26a) prior to the division into (26b)/(26c). In that case, an even greater proportion of the MAN and acetone introduced would be removed from the process than described hereinafter.

In the extraction (P), the aqueous product stream (26b) was treated with the aid of the organic pump distillate (23d) as extractant, in order to reduce the residual content of MMA in stream (26b) prior to the discharge. For this purpose, stream (26b) was fed in at the top of the disc extraction column (P), and stream (23d) at the bottom. In (P), an aqueous raffinate (28b) at the bottom of the column (P) and an organic, methyl methacrylate-enriched extract stream (28a) were obtained. The raffinate stream (28b) contained water, methanol, acetone, methyl methacrylate, MAN and low and high boilers. Raffinate stream (28b) was then blended with the waste acid stream (11) and discharged from the process in the form of the resulting stream (27). MAN and acetone were discharged from the process via (28b). The extract stream (28a) contained methyl methacrylate, acetone, methanol, MAN and further low and high boilers, and was recycled continuously into the reaction zone of the esterification (E). Amounts and compositions are described in Table 3.

The same workup of the bottom product (22b) from the azeotrope column (L) was effected in the same way in Examples A1, A2 and A3.

The low boiler-free bottom product (22b) obtained in the azeotrope column (L), for further purification, was subjected to a reduced pressure rectification (0) that worked at 180 mbar(a) and had a rectifying section and stripping section. Stream (22b) was applied to the middle of the purifying column (O), and this was separated, in accordance with the equilibrium established, into a pure distillate phase (25a) and a high boiler-enriched bottom stream (25b).

The bottom stream (25b) that contained methyl methacrylate, methacrylic acid and high boilers was recycled continuously into the esterification reaction step (E).

The vapour stream obtained was fully condensed in (O). The offgas obtained here at about 2 m³/h was fed to process step (M). The distillate was divided in accordance with the reflux ratio required, such that the amounts of pure MMA product (25a) reported in Table 3 were obtained with >99.9% by weight purity. The pure MMA product (25a) contained acetone and MAN in the amounts reported in Table 3. MAN and acetone were removed from the process in the MMA product stream.

1d. Process Conditions and Results

TABLE 1
Examples A1-A3 - data for amidation, conversion and esterification ((A), (B), (C), (D), (E))

| | | | A1* | A2 | A3* |
|---|---|---|---|---|---|
| | ACH | kg/h | 3750 | 3750 | 3750 |
| (1b) | ACH | kg/h | 1250 | 1250 | 1250 |
| (1a) + (1b) | ACH tot. | kg/h | 5000 | 5000 | 5000 |
| (1a):(1b) | | kg/kg | 75:25 | 75:25 | 75:25 |
| (1a) + (1b) | ACH conc. | % by wt. | 98.8 | 98.8 | 98.8 |
| | H₂SO₄ conc. | % by wt. | 100.3 | 99.7 | 100.3 |
| | Total H₂SO₄ | kg/h | 8100 | 8100 | 8100 |
| (A) | H₂SO₄/ACH | kg/kg | 2.16 | 2.16 | 2.16 |
| (A) + (B) | H₂SO₄/ACH | kg/kg | 1.62 | 1.62 | 1.62 |
| | Total | m³/h | 60 | 50 | 60 |
| (9a) | Total | m³/h | 75 | 65 | 75 |
| (9a) | Acetone | g/m³ | 19.0 | 11.0 | 19.0 |
| (9a) | MAN | g/m³ | 2.0 | 1.1 | 2.0 |
| (9a) | Acetone | kg/h | 2 | 1 | 2 |
| (9a) | MAN | kg/h | 0.2 | 0.1 | 0.2 |
| (8) | Total | kg/h | 12 914 | 13 015 | 12 914 |
| | MAA | % by wt. | 35.6 | 35.4 | 35.6 |
| | MA | % by wt. | 0.4 | 1.0 | 0.4 |
| (8) | HIBAm | % by wt. | 0.4 | 0.4 | 0.4 |
| (8) | Acetone | % by wt. | 0.25 | 0.2 | 0.25 |
| (8) | MAN | ppm | 500 | 354 | 500 |
| (8) | MAA + MA | kg/h | 4649 | 4734 | 4649 |
| | (reactants for | | | | |
| | esterification) | | | | |
| (8) + (9a) | Acetone | kg/h | 34 | 26.5 | 34 |
| (8) + (9a) | MAN | kg/h | 6.5 | 4.7 | 6.5 |
| Amide yield | MAA + MA based | % | 93.0 | 94.7 | 93.0 |
| | on ACH | | | | |
| (29) | Total | kg/h | 590 | 590 | 590 |
| (10a) | Total | kg/h | 1540 | 1570 | 1540 |
| (16d) | Total | kg/h | 500 | 500 | 500 |
| (16a) + (16 c) | Water | kg/h | 1700 | 1700 | 1700 |
| MAN | MAN decrease | kg/h | 1.5 | 1.0 | 1.5 |
| (hydrol.) | in (E) | | | | |
| (11) | Total | kg/h | 11 504 | 11 410 | 11 515 |
| (11) | MAN | ppm | 110 | 60 | 100 |
| (11) | TOC | % by wt. | 2.3 | 1.5 | 2.4 |
| (11) | Solid-state TOC | % by wt. | 0.2 | 0.1 | 0.2 |
| (11) | Sulfo acetone in | ppm | 470 | 360 | 465 |
| | TOC | | | | |
| (11) | Acetone | kg/h | 5.0 | 4.0 | 5.0 |
| (11) | MAN | kg/h | 1.2 | 0.65 | 1.1 |

*comparative example

TABLE 2
Examples A1-A3 - data for workup/prepurification (F), (G), (H))

| | | | A1* | A2 | A3* |
|---|---|---|---|---|---|
| (12) | Total | kg/h | 10810 | 9875 | 10698 |
| (12) | MMA | % by wt. | 67 | 74 | 63 |
| (12) | Water | % by wt. | 22 | 16 | 22 |
| (12) | MeOH | % by wt. | 6.5 | 5 | 6.7 |
| (12) | MA | % by wt. | 3 | 3 | 2.8 |
| (12) | Acetone | % by wt. | 1 | 0.8 | 1 |
| (12) | MAN | % by wt. | 0.15 | 0.09 | 0.1 |
| (30) | Total | kg/h | 83 | 74 | 85 |
| (30) | Acetone | kg/h | 1 | 0.5 | 1 |
| (30) | MAN | kg/h | 0.1 | 0.1 | 0.1 |
| (14a) | Total | kg/h | 10 105 | 9080 | 10 081 |
| (15a) | Total | kg/h | 8156 | 7,638 | 8,120 |
| (15a) | MMA | % by wt. | 83.9 | 88.8 | 84.1 |
| (15a) | Water | % by wt. | 4.1 | 2.9 | 4.1 |
| (15a) | MeOH | % by wt. | 5.4 | 3.4 | 5.4 |
| (15a) | MA | % by wt. | 1.8 | 1.7 | 1.8 |
| (15a) | Acetone | % by wt. | 2.1 | 1.1 | 1.9 |
| (15a) | MAN | % by wt. | 0.4 | 0.1 | 0.3 |
| (15b) | Total | kg/h | 1,949 | 1,447 | 1960 |
| (18) | Total | kg/h | 3,251 | 2,583 | 3261 |
| (18) | Water | % by wt. | 75.0 | 82.0 | 74.7 |
| (18) | MeOH | % by wt. | 17.0 | 12.5 | 17.8 |
| (18) | MMA | % by wt. | 4.0 | 3.4 | 5.0 |
| (18) | MA | % by wt. | 0.4 | 0.2 | 0.4 |
| (18) | Acetone | % by wt. | 1.5 | 0.8 | 1.4 |
| (18) | MAN | ppm | 470 | 100 | 330 |
| (17a) | Total | kg/h | 7,754 | 7,398 | 7,719 |
| (17a) | MMA | % by wt. | 89.5 | 91.2 | 87.7 |
| (17a) | Water | % by wt. | 2.9 | 2.3 | 3.0 |
| (17a) | MeOH | % by wt. | 1.2 | 1.9 | 3.0 |
| (17a) | MA | % by wt. | 1.8 | 1.4 | 1.8 |
| (17a) | Acetone | % by wt. | 1.9 | 1.0 | 1.7 |
| (17a) | MAN | % by wt. | 0.4 | 0.1 | 0.3 |

*comparative example

TABLE 3
Examples A1-A3 - data for workup/fine
purification (L), (O), (M), (N), (P)

| | | | A1* | A2 | A3* |
|---|---|---|---|---|---|
| (22b) | Bottoms (L) | kg/h | // | // | // |
| (22b) | MMA | % by wt. | 96.0 | 96.7 | 95.8 |
| (22b) | MA | % by wt. | 2.0 | 1.5 | 2.0 |
| (22b) | High boilers | % by wt. | <2.0 | 1.8 | <2.0 |
| (22b) | Acetone | ppm | 10 | <10 | 9 |
| (22b) | MAN | ppm | 138 | 37 | 98 |
| (23a) | Condens. | kg/h | 7184 | 8238 | 7282 |
| | vapour | | | | |
| (23a) | MMA | % by wt. | 61 | 75 | 64 |
| (23a) | Water | % by wt. | 8.1 | 5.4 | 7.8 |
| (23a) | Acetone | % by wt. | 11.5 | 7.3 | 10.6 |
| (23a) | MAN | % by wt. | 8.4 | 5.6 | 7.4 |
| (23a) | MAN | kg/h | 603 | 461 | — |
| (23d) | PK | kg/h | // | // | // |
| (23d) | Low boilers | % by wt. | 33 | >30 | 33 |
| (23d) | MMA | % by wt. | 30 | 40 | 32 |
| (23d) | MeOH | % by wt. | 15 | 11 | 15 |
| (23d) | Water | % by wt. | 1.4 | 1.4 | 1.4 |
| (23d) | Acetone | % by wt. | 17.0 | 11.0 | 16.2 |
| (23d) | MAN | % by wt. | 5.9 | 3.0 | 5.2 |
| (23e) | Offgas | kg/h | 28 | 23 | 27 |
| (23e) | Acetone | kg/h | 1.0 | 1.0 | 1.1 |
| (23e) | MAN | kg/h | 0.1 | 0.1 | 0.1 |
| (24a) | Total | kg/h | 6428 | 7795 | 6553 |
| (24a) | Water | % by wt. | 5.5 | 3.5 | 5.3 |
| (24a) | MeOH | % by wt. | 6.3 | 3.6 | 6.1 |
| (24a) | MMA | % by wt. | 66.7 | 78.6 | 70.0 |
| (24a) | Acetone | % by wt. | 10.7 | 6.9 | 9.9 |
| (24a) | MAN | % by wt. | 8.9 | 5.8 | 8.1 |
| (24a) | MAN | kg/h | 572 | 452 | — |
| (24b) | Total | kg/h | 1,355 | 1,043 | 1,328 |
| (24b) | Water | % by wt. | 60.5 | 73.7 | 61.9 |
| (24b) | MeOH | % by wt. | 18.6 | 13.8 | 18.5 |
| (24b) | MMA | % by wt. | 10.5 | 4.5 | 10.0 |
| (24b) | Acetone | % by wt. | 11.0 | 6.4 | 9.6 |
| (24b) | MAN | % by wt. | 1.9 | 0.8 | 1.7 |
| (24b) | MAN | kg/h | 25.7 | 8 | 22.5 |
| (26a) | Total | kg/h | — | — | 266 |
| (26a) | MMA | kg/h | — | — | 26.8 |
| (26a) | Acetone | kg/h | — | — | 25.5 |
| (26a) | MAN | kg/h | — | — | 2.75 |
| (26b) | Total | kg/h | 271 | 522 | — |
| (26c) | Total | kg/h | 1,084 | 522 | 1063 |
| (28a) | Total | kg/h | 72 | 66 | — |
| (28a) | Acetone | % by wt. | 19 | 14.5 | — |
| (28a) | MAN | % by wt. | 3.2 | 2.0 | — |
| (28a) | MAN | kg/h | 2.3 | 1.3 | — |
| (28b) | Total | kg/h | 252 | 508 | — |
| (28b) | Water | % by wt. | 63.0 | 76.0 | — |
| (28b) | MeOH | % by wt. | 19.6 | 14.0 | — |
| (28b) | MMA | % by wt. | 6.7 | 2.3 | — |
| (28b) | Acetone | % by wt. | 9.3 | 4.0 | — |
| (28b) | MAN | % by wt. | 1.1 | 0.5 | — |
| (28b) | Acetone | kg/h | 23 | 20 | — |
| (28b) | MAN | kg/h | 2.65 | 2.7 | — |
| (25a) | Total | kg/h | 5453 | 5534 | 5440 |
| (25a) | MMA | % by wt. | >99.9 | >99.9 | >99.9 |
| (25a) | Acetone | ppm | 10 | <10 | 10 |
| (25a) | MAN | ppm | 176 | 47 | 118 |
| (25a) | Acetone | kg/h | 0.2 | <0.1 | 0.15 |
| (25a) | MAN | kg/h | 0.96 | 0.26 | 0.65 |

*comparative example,
— = absent

The results in Tables 1 to 3 show that, in Inventive Example A2 using sulfuric acid with a concentration of 99.7% by weight (0.3% by weight of water), lower proportions of MAN and acetone are obtained in the amidation and conversion (see stream (8) in Table 1) and are fed into the esterification than by comparison with Examples A1 and A3 using sulfuric acid having a concentration of 100.3% by weight (0.3% by weight of free SO₃).

In addition to the reduced sulfuric acid concentration, in Inventive Example A2, an elevated amount of aqueous phase from phase separator (N) is discharged (ratio of 26c/26b=50:50 vs. 80:20 in Comparative Example A1).

Moreover, the inventive procedure according to Example A2 has the effect that the pure MMA product has much lower contamination with MAN than is the case in the comparative example according to the prior art. The proportion of MAN and acetone in the MMA end product (25a) in Example A2 is distinctly reduced compared to Example A1 (see Table 3).

Table 4 below compiles the streams of matter from Table 3 relating to the enrichment of MAN in steps (L), (M) and (N), and relating to the recycling of MAN and the discharge of MAN via (M) and (P) for Examples A1 and A3 (comparative examples) and A2 (inventive).

TABLE 4
Overview of selected process data and overview of MAN
recycling and MAN discharge in (L), (M) (N) and (P)

| Example | | | A1* | A2 | A3* |
|---|---|---|---|---|---|
| Sulfuric acid conc. (2) | | % by wt. | 100.3 | 99.7 | 100.3 |
| (26c)/(26b) ratio | | kg/kg | 80/20 | 50/50 | — |
| (26c)/(26a) ratio | | kg/kg | — | — | 80/20 |
| Aqueous phase to esterification (recycle) | Total | kg/h | 1,084 | 522 | 1,063 |
| (26c) | amount | | | | |
| Aqueous phase to esterification (recycle) | Amount | kg/h | 20.6 | 4.0 | 16.4 |
| (26c) | of MAN | | | | |
| Total recycle rate into the esterification | Total | kg/h | 1,156 | 574 | 1,063 |
| (26c) + (28a) or (26c) only | amount | | | | |
| Total recycle rate into the esterification | Amount | kg/h | 23.0 | 5.3 | 19.8 |
| (26c) + (28a) | of MAN | | | | |
| Raffinate to cleavage acid (discharge | Total | kg/h | 252 | 508 | — |
| after extraction) (28b) | amount | | | | |
| Discharge of aqueous phase after phase | Total | kg/h | — | — | 266 |
| separator II (26a) | | | | | |
| MAN discharge in (28b) or (26a) | Amount | kg/h | 2.65 | 2.70 | 2.75 |
| | of MAN | | | | |
| Quotient (MAN recycling/MAN | | kg/kg | 8.7 | 2.0 | 7.3 |
| discharge) | | | | | |
| MMA loss in discharge (28b) or (26a) | Amount | kg/h | 16.9 | 11.6 | 26.8 |
| | of MMA | | | | |
| Total MMA yield loss via discharged | | % | 0.31 | 0.20 | 0.5 |
| MMA | | | | | |
| Total MMA yield | MMA | % | 90.3 | 91.6 | 90.1 |
| | based on | | | | |
| | ACH | | | | |

*comparative example

On the basis of the values in Table 4, it is found that, in Comparative Example A1, a greater amount of MAN as troublesome by-product in the process (esterification reactor E) is recycled (via (28a)) and less troublesome MAN can be discharged from the process (via (28b)) compared to Inventive example A2. The quotient of MAN (recycled) to MAN (discharged) is thus much lower in Inventive Example A2 and hence more advantageous than in Comparative Example A1. The loss of MMA via the discharge of aqueous phase (28b) is distinctly increased in Comparative Examples A1 and A3 compared to Inventive Example A2, with a smaller MMA loss through use of the extraction in Comparative Example A1 than in Comparative Example A3. On account of the lower enrichment of acetone and MAN in Inventive Example A2, the MMA loss is at its lowest even though the amount of aqueous phase discharged is greater than in Comparative Examples A1 and A3.

In addition, it is shown experimentally that the discharged cleavage acid (11) that has optionally been sent to a further utilization is less contaminated with sulfonated acetone (see Table 1).

### Example B1 According to FIG. 3 (Variant B)

The preparation of methyl methacrylate comprising the reaction of acetone cyanohydrin with sulfuric acid in the amidation/conversion (A, B, C, D), the subsequent esterification (E) with methanol, and distillative and extractive workup (F, I, J, K, L, O, M) of the methyl methacrylate product was effected by the embodiment according to FIG. 3 as described above (variant B).

A mass balance and and assessment of the discharge of MAN and acetone via the total offgas (11), output air streams (21a) and (9b), and via the MMA product (25a) was effected (see flow diagram according to FIG. 3).

An inventive example (Example B1) using sulfuric acid having a concentration in the region of 99.7% by weight (0.3% by weight of water) is described hereinafter. The example describes the preparation of methyl methacrylate having the claimed features, with achievement of a high overall yield of MMA, characterized by reduced MAN formation in the amidation and conversion. By the exclusively distillative workup of the MMA product (without extraction) and discharge of by-products such as acetone in the offgas, it is possible to achieve a high overall MMA yield with moderate MAN content analogously to Example 2A.

The performance of the process according to the invention as per FIG. 3 (variant B) is described hereinafter.

2a. Reaction Stages (A), (B), (C), (D) and (E)

The amidation (A) and (B), the conversion (C) and gas separation (D) were performed as described above in Example 1, with the differences specified hereinafter:
The gas space of the amidation reactors (A) and (B) was operated at about 970 mbara, at slightly reduced pressure.
The intermediate vessel (D) was operated at a temperature of 100° C. The mixture (7) was cooled in the region of the vessel (D) by means of a water-cooled heat exchanger through which an amide circulation stream connected to the vessel (D) flowed.
Suitable interconnection of the reactant streams (10a, 10b, 16b, 16d, 29) and of the circulation streams (13, 20c, 21b, 25b) achieved a local stoichiometric excess of methanol and water based on methacrylamide and methacrylic acid in each of the six esterification tanks.
An overall offgas (9b) was obtained in the gas separator (D), which contained acetone and methacrylamide and was discharged continuously from the process.

The amounts and compositions are collated in Table 5.

2b. Prepurification (F), (I), (J)

According to the vapour/liquid equilibrium of the reaction mixtures, the vapour stream (12) left the esterification with a heteroazeotropic composition comprising MMA, water, MeOH, MA, acetone and MAN as reported in Table 6.

Vapour stream (12) was subsequently subjected to a countercurrent distillation (F), wherein the vaporous stream (12) was applied in the bottom region of a column (F). The distillation (F) was operated at a slightly elevated pressure of 100 mbar(g).

Vapour stream (12) was partially condensed at the top of the column, and an organic, liquid side stream (19c) from column (I) was applied as subcooled reflux. By this procedure, a methacrylic acid-containing bottom stream (13) was obtained, which is recycled directly into the esterification (E). The bottom stream contains MMA. MA and further high boilers according to Table 6. At the top of the column (F), a vaporous heteroazeotropic vapour stream (14b) was obtained, which was sent to further distillative separation in the middle of a rectification column (I). The heteroazeotropic stream (14b) contained MMA, MeOH, water, acetone and MAN according to Table 6.

The primary stripper column (I) was heated with hot steam and operated at a slightly elevated pressure of 100 mbarg. A heteroazeotropic mixture of methyl methacrylate, water, methanol, acetone and MAN that has been depleted of low boilers in the bottom stream (19b) was separated from a low boiler-enriched top stream (19a). It was possible to control the low boiler content and hence the content of acetone and MAN among others by the energy input in the evaporator.

At the top of the column (I), the vapour stream obtained was partially condensed, the condensate was returned to column (I) as liquid reflux, and the remaining low boiler-containing vapour phase (19a) that comprised MeOH, MMA, water, acetone and MAN, and also further low boilers according to Table 6 was sent to an offgas scrubbing (J). The enrichment of low boilers in the top region of the column (I) was controlled here via the temperature in the partial condenser.

A portion of the liquid low-boiling mixture at the top of column (I) was removed continuously from the column (I) in the form of a liquid side stream draw (19c). Side stream draw (19c) contained MMA, MeOH, water, acetone and MAN according to Table 6 and, in subcooled form, served as reflux for scrubbing column (F).

The vapour stream (19a) was then fed together with offgas (23b) from the azeotrope column (L) to the bottom region of an offgas scrubbing column (J) that had a partial condenser at the top. The vapours fed in (19a) ascended within column (J) and were scrubbed by the high boiler-containing reflux formed at the top by condensation, so as to obtain an MMA-depleted offgas (21a) at the top of the column (J) and a liquid, MMA-enriched mixture (21b) at the bottom of column (J). The scrubbing process in (J) was supported by addition of fresh methanol (10b) that was applied at the top of column (J). The bottom stream (21b) from (J), comprising MeOH and MMA, was sent to the esterification (E). The offgas (21a) comprised low boilers and inerts, MeOH, acetone and MAN, and was sent to an incineration. Amounts and compositions are reported in Table 6.

The heteroazeotropic bottom stream (19b) obtained in the primary stripper (1), for separation of the liquid phases, was first cooled to 30° C. and then sent to the phase separation vessel (K). In addition to stream (19b), in the phase separator (K), an MMA-containing heteroazeotropic distillate (23c) was sent to the condensation/vacuum unit (M). The aqueous heavy phase (20b) contained water, methanol, MMA, acetone, MA and high and low boilers according to Table 6, and was sent continuously to a mixing zone. Here, the addition of deionized water (16b) afforded a diluted stream (20c) that was fed fully to the esterification (E). The organic light phase (20a) contained MMA, water, methanol, acetone, MA, MAN and high and low boilers according to Table 6, and was sent to the fine purification in the azeotrope column (L).

2c. Fine Purification (L), (O), (M)

The organic light phase (20a), for removal of low boilers, was sent to the top region of an azeotrope column (L) operated under reduced pressure, which was indirectly heated with hot steam and separates a low boiler-enriched heteroazeotropic vapour stream (22a) from a methyl methacrylate-enriched bottom stream (22b).

The vapour stream (22a) contained MMA, water, MeOH, acetone and MAN according to Table 7. The bottom stream (22b) that was freed of low boilers in the desired manner contained MMA, MA, high boilers and the MAN and acetone by-products according to Table 7.

The bottom product (22b) obtained in the azeotrope column (L), for further purification, was subjected to a reduced pressure rectification (O) that was performed at 180 mbar(a) and was characterized by a rectifying section and stripping section. Stream (22b) was applied to the middle of the purifying column (O), which gave, in accordance with the equilibrium established, a pure MMA product (25a) and a high boiler-enriched bottom stream (25b). The resultant vapour stream was fully condensed, the resultant offgas of 4 m³/h was sent fully to process step (M), and the distillate was divided in accordance with the required reflux ratio such that, based on the esterification line (E), the amount of MMA with >99.9% purity specified in Table 7 was obtained. The proportion of acetone and MAN, and the amounts discharged, are stated in Table 7.

The resultant bottom stream (25b) containing MMA, MA and high boilers according to Table 7 was returned continuously to reaction step (E).

2d. Process Conditions and Results

TABLE 5
Example B1 (inventive) - data for amidation, conversion
and esterification ((A), (B), (C), (D), (E))

| | | | |
|---|---|---|---|
| (1a) | ACH | kg/h | 6,845 |
| (1b) | ACH | kg/h | 3,685 |
| (1a) + (1b) | ACH tot. | kg/h | 10,530 |
| (1a):(1b) | | kg/kg | 65:35 |
| (1a) + (1b) | ACH conc. | % by wt. | 99.0 |
| (2) | H₂SO₄ conc. | % by wt. | 99.7 |
| (2) | Total | kg/h | 17,060 |
| (A) | H₂SO₄/ACH | kg/kg | 2.55 |
| (A) + (B) | H₂SO₄/ACH | kg/kg | 1.62 |
| (5b) | Total | m³/h | 85 |
| (9b) | Total | m³/h | 105 |
| (9b) | Acetone | g/m³ | 15 |
| (9b) | MAN | g/m³ | 1 |
| (9b) | Acetone | kg/h | 2 |
| (9b) | MAN | kg/h | 0.1 |
| (8) | Total | kg/h | 27,475 |
| (8) | MAA | % by wt. | 35.7 |
| (8) | MA | % by wt. | 0.9 |
| (8) | HIBAm | % by wt. | 0.2 |
| (8) | Acetone | % by wt. | 0.2 |
| (8) | MAN | ppm | 322 |
| (8) | Acetone | kg/h | 53 |
| (8) | MAN | kg/h | 8.6 |
| (8) | MAA + MA (reactants for | kg/h | 10,080 |
| | esterification) | | |
| Amide yield | MAA + MA based on ACH | % | 95.7 |
| (29) | Total (MMA/MeOH) | kg/h | 700 |
| (10a) | Total (MeOH) | kg/h | 3,300 |
| (16d) | Total (direct steam) | kg/h | 550 |
| (16b) | Water | kg/h | 3450 |
| MAN (hydrol.) | MAN decrease in (E) | kg/h | 4.8 |
| (11) | Total | kg/h | 23570 |
| (11) | TOC | % by wt. | 3.2 |
| (11) | Solid-state TOC | % by wt. | 0.2 |
| TOC/(11) | Sulfo acetone | ppm | 265 |
| TOC/(11) | MAN | ppm | 120 |
| (11) | Acetone | kg/h | 7 |
| (11) | MAN | kg/h | 2.9 |

TABLE 6
Example B1 (inventive) - data for workup/prepurification
(F), (I), (J))

| | | | |
|---|---|---|---|
| (12) | Total | kg/h | 32304 |
| (12) | MMA | % by wt. | 87.9 |
| (12) | Water | % by wt. | 5.3 |
| (12) | MeOH | % by wt. | 3.6 |
| (12) | MA | % by wt. | 1.3 |
| (12) | Acetone | % by wt. | 0.6 |
| (12) | MAN | ppm | 52 |
| (14b) | Total | kg/h | 23445 |
| (14b) | MMA | % by wt. | 73.9 |
| (14b) | Water | % by wt. | 10.3 |
| (14b) | MeOH | % by wt. | 13.8 |
| (14b) | Acetone | % by wt. | 1.5 |
| (14b) | MAN | ppm | 105 |
| (19a) | Total | kg/h | 1,684 |
| (19a) | MeOH | % by wt. | >60.0 |
| (19a) | MMA | % by wt. | 25.0 |
| (19a) | Water | % by wt. | 2.0 |
| (19a) | Acetone | % by wt. | 9.3 |
| (19a) | MAN | ppm | 179 |
| (19c) | Total | kg/h | — |
| (19c) | MMA | % by wt. | 60 |
| (19c) | Water | % by wt. | 10 |
| (19c) | MeOH | % by wt. | 28 |
| (19c) | Acetone | % by wt. | 2 |
| (19c) | MAN | ppm | 200 |
| (21a) | Total | kg/h | 220 |
| (21a) | Low boilers/inerts | % by wt. | >60 |
| (21a) | MeOH | % by wt. | 20 |
| (21a) | Acetone | % by wt. | 18 |
| (21a) | MAN | ppm | 330 |
| (21a) | Acetone | kg/h | 37 |
| (21a) | MAN | kg/h | 0.1 |
| (20b) | Total | kg/h | 1,833 |
| (20b) | Water | % by wt. | 88.0 |
| (20b) | MeOH | % by wt. | 8.1 |
| (20b) | MMA | % by wt. | 2.9 |
| (20b) | MA | % by wt. | 0.02 |
| (20b) | Acetone | % by wt. | 0.03 |
| (20b) | MAN | ppm | 15 |
| (20a) | Total | kg/h | 17,998 |
| (20a) | MMA | % by wt. | 95.7 |
| (20a) | Water | % by wt. | 2.2 |
| (20a) | MeOH | % by wt. | 1.6 |
| (20a) | MA | % by wt. | 0.1 |
| (20a) | Acetone | ppm | 250 |
| (20a) | MAN | ppm | 127 |

TABLE 7
Examples B1 (inventive) - data for workup/fine
purification (L), (O), (M)

| | | | |
|---|---|---|---|
| (22a) | Total | kg/h | 5,814 |
| (22a) | MMA | % by wt. | 88.6 |
| (22a) | Water | % by wt. | 6.8 |
| (22a) | MeOH | % by wt. | 5.0 |
| (22a) | Acetone | % by wt. | 0.08 |
| (22a) | MAN | ppm | 280 |
| (22b) | Total | kg/h | — |
| (22b) | MMA | % by wt. | >99.0 |
| (22b) | MA | % by wt. | 0.2 |
| (22b) | High boilers | % by wt. | 0.3 |
| (22b) | Acetone | ppm | <10 |
| (22b) | MAN | ppm | 64 |
| (23c) | Total | kg/h | ~5780 |
| (23b) | Total offgas | kg/h | — |
| (25a) | Total pure MMA | kg/h | 11,390 |
| (25a) | MMA | % by wt. | >99.9 |
| (25a) | Acetone | ppm | <10 |
| (25a) | MAN | ppm | 70 |
| (25a) | Acetone | kg/h | <0.1 |
| (25a) | MAN | kg/h | 0.8 |
| MMA yield | MMA based on | % | 91.1 |
| | ACH | | |
| (25b) | Bottoms | kg/h | 800 |
| (25b) | MMA | % by wt. | 87.0 |
| (25b) | MA | % by wt. | 4.0 |
| (25b) | High boilers | % by wt. | 9.0 |

The results in Tables 4 to 6 show that, in Inventive Example B1 using sulfuric acid having a concentration of 99.7% by weight (0.3% by weight of water), a small proportion of MAN and acetone is obtained in the amidation and conversion and is fed into the esterification. In association with an exclusively distillative purification of the crude product and the gaseous discharge of secondary components, the effect of this procedure is that the pure MMA product has much lower contamination with MAN.

1: A process for preparing alkyl methacrylate, comprising:
a. reacting acetone cyanohydrin and sulfuric acid in one or more reactors I in a first reaction stage for amidation at a temperature in the range from 70 to 130° C., to obtain a first reaction mixture comprising sulfoxyisobutyramide and methacrylamide;
b. converting the first reaction mixture by heating to a temperature in the range from 130 to 200° C. in one or more reactors II in a second reaction stage for conversion, to obtain a second reaction mixture comprising predominantly the methacrylamide and the sulfuric acid;
c. reacting the second reaction mixture with alcohol and water, in one or more reactors III in a third reaction stage for esterification, to obtain a third reaction mixture comprising the alkyl methacrylate; and
d. separating the alkyl methacrylate from the third reaction mixture obtained from the third reaction stage;
wherein the sulfuric acid used in the first reaction stage has a concentration in the range from 98.0% by weight to 100.0% by weight;
wherein the separation the alkyl methacrylate from the third reaction mixture comprises at least two distillation steps in which methacrylonitrile and acetone by-products are obtained at least partly in a tops fraction as a water-containing heteroazeotrope,
wherein the water-containing heteroazeotrope comprising the methacrylonitrile and the acetone from at least one of the at least two distillation steps is at least partly discharged from the process, and
wherein at least one stream comprising the methacrylonitrile and the acetone is at least partly recycled into the third reaction stage.

2: The process according to claim 1, wherein the at least one stream comprising the methacrylonitrile and the acetone which is at least partly recycled into the third reaction stage is a water-containing heteroazeotrope comprising the methacrylonitrile and the acetone from at least one of the at least two distillation steps.

3: The process according to claim 1, wherein at least one aqueous phase which is obtained by condensation and phase separation of the water-containing heteroazeotrope from at least one of the at least two distillation steps is recycled fully or partly into the third reaction stage, where the at least one aqueous phase is contacted with the second reaction mixture comprising predominantly the methacrylamide and the sulfuric acid.

4: The process according to claim 1, wherein at least one aqueous phase which is obtained by condensation and phase separation of the water-containing heteroazeotrope from at least one of the at least two distillation steps is discharged fully or partly from the process, optionally after an extraction step.

5: The process according to claim 1, wherein the water-containing heteroazeotrope from at least one of the at least two distillation steps is discharged fully or partly from the process, at least partly in the form of a gaseous stream, optionally after a scrubbing step.

6: The process according to claim 1, wherein the separation of the alkyl methacrylate from the third reaction mixture comprises at least one phase separation step in which the water-containing heteroazeotrope from at least one of the at least two distillation steps is separated into an aqueous phase comprising the methacrylonitrile and the acetone, and an organic phase comprising predominantly the alkyl methacrylate,
wherein the aqueous phase is partly discharged from the process and/or partly recycled into the third reaction stage, and
wherein the organic phase comprising predominantly the alkyl methacrylate is recycled fully or partly into the at least one of the at least two distillation steps.

7: The process according to claim 1, wherein the third reaction mixture obtained in the third reaction stage is evaporated continuously, wherein a resultant vapour stream is fed to a first distillation step K1 in which a tops fraction comprising the alkyl methacrylate, water and the alcohol, and a bottoms fraction comprising higher-boiling components are obtained, and
wherein the bottoms fraction is recycled fully or partly into the third reaction stage.

8: The process according to claim 1, wherein the separation of the alkyl methacrylate from the third reaction mixture comprises
(i) first distilling the third reaction mixture obtained in the third reaction stage in a first distillation step K1, to obtain a first water-containing heteroazeotrope comprising the methacrylonitrile and the acetone as a tops fraction;
(ii) separating the first water-containing heteroazeotrope as condensate in a phase separation step in a phase separator I, into an aqueous phase WP-1 and an organic phase OP-1 comprising a predominant portion of the alkyl methacrylate;
(iii) guiding the organic phase OP-1 into a second distillation step K2, wherein a further tops fraction obtained is a second water-containing heteroazeotrope comprising the methacrylonitrile and the acetone;
(iv) separating at least a portion of the second water-containing heteroazeotrope in a phase separation step in a phase separator II, into an aqueous phase WP-2 comprising the methacrylonitrile and the acetone, and an organic phase OP-2,
wherein the organic phase OP-2 is recycled fully or partly into the second distillation step K2, and
wherein the aqueous phase WP-2 comprising the methacrylonitrile and the acetone is partly recycled into the third reaction stage and partly discharged from the process, optionally after an extraction step.

9: The process according to claim 8, wherein the aqueous phase WP-1 is recycled fully or partly into the third reaction stage and the organic phase OP-1 comprising the predominant portion of the alkyl methacrylate is subjected to an extraction using water as extractant,
wherein an aqueous phase of the extraction is recycled into the third reaction stage and a organic phase of the extraction is guided into the second distillation step K2.

10: The process according to claim 8, wherein a portion of the aqueous phase WP-2 comprising the methacrylonitrile and the acetone is subjected to an extraction to obtain an aqueous phase WP-3 and an organic phase OP-3,
wherein the aqueous phase WP-3 is discharged fully or partly from the process, and
wherein the organic phase OP-3 is recycled fully or partly into the third reaction stage.

11: The process according to claim 8, wherein a portion of the aqueous phase WP-2 comprising the methacrylonitrile and acetone is subjected to an extraction to obtain an aqueous phase WP-3 and an organic phase OP-3,
wherein the aqueous phase WP-3 is subjected to a further distillation step K4,
wherein a tops fraction comprising the methacrylonitrile is obtained in distillation step K4 and discharged from the process,
wherein a bottoms fraction comprising water is obtained in distillation step K4 and recycled fully or partly into the extraction, and
wherein the organic phase OP-3 is recycled fully or partly into the third reaction stage.

12: The process according to claim 1, wherein the separation of the alkyl methacrylate from the third reaction mixture comprises
(i) first distilling the third reaction mixture obtained in the third reaction stage in a first distillation step K1, to obtain a first water-containing heteroazeotrope comprising the methacrylonitrile and the acetone as a tops fraction;
(ii) guiding the first water-containing heteroazeotrope as a vapour stream into a further distillation step K4, in which a further water-containing heteroazeotrope comprising the methacrylonitrile and the acetone is obtained as a tops fraction, and a bottoms fraction comprising the alkyl methacrylate is obtained,
(iii) discharging the tops fraction from distillation step K4, optionally after a scrubbing step, fully or partly from the process in the form of a gaseous stream,
(iv) separating the bottoms fraction from distillation step K4 in a phase separation step in phase separator II, into an aqueous phase WP-2 comprising the methacrylonitrile and acetone, and an organic phase OP-2, wherein the aqueous phase WP-2 comprising the methacrylonitrile and the acetone is recycled fully or partly into the third reaction stage, and
(v) guiding the organic phase WP-2 fully or partly into a second distillation step K2, in which the tops fraction obtained is a second water-containing heteroazeotrope comprising the methacrylonitrile and the acetone, which is condensed fully or partly and guided into the phase separation step in the phase separator II according to (iv).

13: The process according to claim 1, wherein the second reaction mixture contains not more than 3% by weight of methacrylic acid, not more than 1.5% by weight of alpha-hydroxyisobutyramide and not more than 0.3% by weight of the methacrylonitrile, based in each case on the overall amount of the second reaction mixture.

14: The process according to claim 1, wherein the second reaction mixture contains 30% to 40% by weight of the methacrylamide, based on the overall amount of the second reaction mixture.

15: The process according to claim 1, wherein the conversion of the acetone cyanohydrin and the sulfuric acid in the first reaction stage is effected in at least two separate reaction zones or in at least two separate reactors.

16: The process according to claim 1, wherein, in the first reaction stage, the conversion of the acetone cyanohydrin (ACH) and the sulfuric acid is effected in at least two separate reactors,
wherein the sulfuric acid and the acetone cyanohydrin are used in a first reactor in a molar ratio of the sulfuric acid to the ACH in the range from 1.6 to 3.0, and
wherein the sulfuric acid and the acetone cyanohydrin are used in a second reactor in a molar ratio of the sulfuric acid to the ACH in the range from 1.2 to 2.0.

17: The process according to claim 1, wherein the reaction mixture of acetone cyanohydrin (ACH) and sulfuric acid in the first reaction mixture includes a total amount of water in the range from 0.1 mol % to 20 mol %, based on an entirety of the ACH supplied to the first reaction stage.

18: The process according to claim 8, wherein a crude alkyl methacrylate product is obtained as a bottoms fraction in the second distillation step K2,
wherein the crude alkyl methacrylate product is purified in a further distillation step K3, to obtain a pure alkyl methacrylate product as a tops fraction, having a methacrylonitrile content in the range from 10 to 300 ppm, based on an amount of the pure alkyl methacrylate product.

19: The process according to claim 1, wherein the process comprises a regeneration of the sulfuric acid, wherein a portion of the third reaction mixture obtained in the third reaction stage and at least one aqueous or organic waste stream comprising sulfuric acid, ammonium hydrogensulfate and sulfonated acetone derivatives that results from discharge of the water-containing heteroazeotrope comprising the methacrylonitrile and the acetone is sent to a thermal regeneration step in which the sulfuric acid is obtained, which is recycled into the first reaction stage.

20: The process according to claim 1, wherein the process comprises obtaining ammonium sulfate, wherein a portion of the third reaction mixture obtained in the third reaction stage and at least one aqueous or organic waste stream comprising sulfuric acid, ammonium hydrogensulfate and sulfonated acetone derivatives that results from discharge of the water-containing heteroazeotrope comprising the methacrylonitrile and the acetone is sent to a thermal regeneration step in which the ammonium sulfate is obtained by crystallization, which is separated off as a by-product.
